(19) United States
(12) Patent Application Publication
(10) Pub. No.: US 2010/0075390 A1
(43) Pub. Date: Mar. 25, 2010

(54) DEVELOPMENT AND USE OF AN IRON-BASED CATALYST FOR IMPLEMENTING AN OXIDATION-REDUCTION PROCESS FOR SUBSTANCES TO BE REDUCED

(75) Inventors: Jean-Marie Genin, Neuviller Sur Mozelle (FR); Christian Ruby, Tomblaine (FR)

Correspondence Address: YOUNG & THOMPSON, 209 Madison Street, Suite 500, Alexandria, VA, 22314, US

(73) Assignee: UNIVERSITE HENRI POINCARE NANCY 1, Nancy (FR)

(21) Appl. No.: 12/525,219

(22) PCT Filed: Jan. 31, 2008
(86) PCT No.: PCT/FR2008/000117

§ 371 Date: Nov. 20, 2009

(30) Foreign Application Priority Data
Jan. 31, 2007 (FR) PCT/FR2007/000178

(51) Int. Cl. C12P 3/00; B01J 23/745; B01J 23/72; B01J 23/66; B01J 23/06; B01J 23/755; B01J 23/34; C12P 1/00; B01J 27/185; C12N 1/12

(52) U.S. Cl. 435/168; 502/338; 502/331; 502/330; 502/328; 502/337; 502/324; 435/41; 502/213; 435/257.1

(57) ABSTRACT

The invention relates to the use of a ferrous ferric oxyhydroxy salt of the dual lamellar hydroxide family as a catalyst, or as a precursor of the catalyst having the same crystalline structure as the catalyst, for implementing an oxidation-reduction method, the ferrous ferric oxyhydroxy salt being used in association with ferri-reducing bacteria capable of reducing Fe^{III} into Fe^{II} in the presence of organic material, in order to reduce a substance (S) into a reduced substance, the redox potential of the S_{reduced}/S couple being higher than that of the Fe^{II}/Fe^{III} couple at the crystallographic sites of Fe^{II}.

The invention relates to the use of a novel iron-based catalyst for implementing an oxidation-reduction process for substances to be reduced in the presence of bacteria.

The ferrous-ferric oxyhydroxy salts are intermediate compounds in the degradation of ferrous materials, which are ultimately converted to rust and so are commonly called green rusts on the basis of their colour.

Ona Nguema et al., 2002, Enviro. Sci. Technol., described the formation in vitro of green rusts by the dissimilatory iron-reducing bacteria Shewanella putrefaciens in the presence of methanoate (HCO₂⁻) as electron donor and of lepidocrocite, ferric oxyhydroxide γ—FeOOH, as electron acceptor, and source of iron. The bacterial activity thus consists in reducing the Fe^{III} ions to Fe^{II} ions while oxidizing the organic matter to carbonate CO₃²⁻, which then allows the green rust, called carbonated rust, to form.

Moreover, bacteria can, for example, reduce nitrates, in two ways: directly or indirectly.

The iron-reducing bacteria permit the reduction of Fe^{III} to Fe^{II}, with the Fe^{III} performing the role of final electron acceptor during bacterial respiration, in the course of which the organic matter is oxidized.

By way of example, the waters from septic tanks is often discharged directly into the environment without treatment, more particularly in the case of a scattered settlement; the same nearly always applies to liquid manure from animal husbandry, which is spread on fields in order to utilize the nitrates that it contains as fertilizer. The problem is that the amount of nitrates contained in the liquid manure from agricultural activity often greatly exceeds the requirements for crop growing. The excess nitrate contained in the liquid manure that is not consumed inevitably leaches into the aquifers and thus contributes to diffuse pollution.

The present invention relates to the use of an iron-based catalyst for the implementation of an oxidation-reduction process.

The present invention also relates to a process for pollution control of a medium to be treated.

Another subject of the invention is to provide a novel product permitting the implementation of a process for pollution control of a medium to be treated.

The present invention relates to the use of at least one lamellar double hydroxide (LDH) as catalyst or as precursor of said catalyst, with the same crystalline structure as that of said catalyst, for the implementation of an oxidation-reduction process, said LDH comprising a divalent cation M²⁺ partially or completely substituted with Fe^{II}, and a trivalent cation T³⁺ optionally substituted with Fe^{III}, of the following general formula:

[M²⁺_{(z)}Fe^{II}_{(1-y-z)}T³⁺ₜFe^{III}_{(y-t)}O₂H₂]ⁿ⁺[(y/n)Aⁿ⁻,mH₂O]ⁿ⁻,

in which:
¼<y<⅓, z<1−y and t<y, Aⁿ⁻ is an anion with charge n, n having the values 1, 2 or 3, in particular 2, m is an integer varying from 1 to 10, in particular from 1 to 4, advantageously 3,
and the ratio x=(y−t)/(1−z−t) can vary from 0 to 1,
said LDH being used in association with iron-reducing bacteria that are able to reduce Fe^{III} to Fe^{II} and in the presence of organic matter, and can be deprotonated to give the following formula:

[M²⁺_{(z)}Fe^{II}_{(1-y-z-w)}T³⁺ₜFe^{III}_{(y-t+w)}O₂H_{2-w}]ⁿ⁺[(y/n)Aⁿ⁻,mH₂O]ⁿ⁻,

in which:
A, y, z, m and n are as above,
w corresponds to the degree of deprotonation of the OH⁻ ions,
and the ratio x=(y−t+w)/(1−z−t) can vary from 0 to 1,
in order to reduce a substance S to a substance S_{reduced}, the redox potential of the pair S_{reduced}/S being greater than that of the pair Fe^{II}/Fe^{III} at the crystallographic sites of the Fe^{II},
x varying essentially in the range from 0.33 to 0.66 after the start-up of the oxidation-reduction process, and without a substantial change in the crystalline structure of the aforesaid LDH.

The LDHs are lamellar compounds displaying considerable anisotropy of their chemical bonds, strong within the hydroxylated lamellae, weaker for the cohesion between the lamellae. This characteristic permits the intercalation of a great variety of chemical species, both inorganic and organic or even biological, enabling the reactivity of the material to be modified.

The term “catalyst” denotes here that the LDH participates chemically in the oxidation-reduction process, and is regenerated in the course of the process, owing to the bacterial activity.

By “catalyst” is meant a functional catalyst.

“Precursor with identical crystalline structure” denotes a LDH with the same crystalline structure as that of the catalyst and where the only difference is in the protonation or deprotonation of the OH⁻ ions.

The catalyst has the formula [M²⁺_{(z)}Fe^{II}_{(1-y-z)}T³⁺ₜFe^{III}_{(y-t)} O₂H₂]ⁿ⁺ [y/n)Aⁿ⁻, m H₂O]ⁿ⁻, in which x varies from 0.33 to 0.66 and the precursor has the same formula in which x can be less than 0.33 or greater than 0.66, and x can reach values of 0 and 1.

The start-up phase of the oxidation-reduction process permits the functional catalyst to be obtained from its precursor, which then has the following formula:

[M²⁺_{(z)}Fe^{II}_{(1-y-z-w)}T³⁺ₜFe^{III}_{(y-t+w)}O₂H_{2-w}]ⁿ⁺[(y/n)Aⁿ⁻,mH₂O]ⁿ⁻

The inventors have shown that the LDH as defined above can serve as a catalyst in oxidation-reduction processes in order to reduce a substance S and that the iron-reducing bacteria are effectively bacteria that thus permit nitrates to be reduced indirectly.

By “0.33” is meant the exact value ⅓.

By “0.66” is meant the exact value ⅔.

The value of x within the LDH corresponds to the ratio Fe^{III}/(Fe^{II}+Fe^{III}) and can be directly measured in situ by Mössbauer spectrometry.

The expression “crystalline structure” denotes that said LDH is in the form of a hexagonal-base prismatic solid with a regular, repeating structure, formed from an ordered stack of atoms, molecules or ions, according to the laws of periodicity of translation called a Bravais lattice.

Transition of the solid from x=0.33 to x=1 can take place continuously by progressive oxidation under conditions of intensive oxidation such as is achieved with hydrogen peroxide H₂O₂. It is a phenomenon of deprotonation within the compound, in the course of which some OH⁻ ions become O²⁻, correspondingly converting Fe^{II} ions to Fe^{III}.

The substance S according to the invention denotes any substance capable of being reduced to a substance S_{reduced} and which corresponds to a pair S_{reduced}/S the redox potential of which is necessarily higher than that of the pair Fe^{II}/Fe^{III} at the crystallographic sites of the Fe^{II}.

The substance S is in particular present in a liquid medium. Said liquid medium is laden with organic matter to a varying degree.

By “oxidation-reduction process” is meant a process involving at least two reactions: an oxidation reaction and a reduction reaction, which involve a transfer of electrons by emission and reception, respectively.

The substance S is brought into contact with the LDH at the moment of initiation of the process or during activation of the catalyst or after the start-up of the catalyst.

By “organic matter” is meant any carbon-containing matter whether or not obtained from living organisms (animal or vegetable), which serves as nutrients for the bacteria.

This organic matter can be of natural origin, such as humic acids or compost, or of artificial origin, such as acetate or methanoate.

The bacteria that are described as iron-reducing, used in the invention, are able to reduce Fe^{III} to Fe^{II}. This reduction is made possible by the respiration of the bacteria, in the course of which organic matter is oxidized, and in which the final electron acceptor is Fe^{III}. The oxidation of the organic matter according to the invention is an enzymatic catalytic oxidation.

The bacteria can originate from the bacterial flora of the soil in the humus or from compost, added as a source of organic matter, or even from an inoculum of bacteria. The inoculum of bacteria can be obtained from bacteria grown in vitro, in lyophilized or frozen form.

The value of x varies during the oxidation-reduction process, and in a novel way, this variation of x takes place in situ, and does not involve any substantial change in the structure of the LDH. In fact, the bacterial reduction can take place without dissolution of the ferric precursor followed by reprecipitation of the LDH.

The expression “without substantial change in its structure” denotes that the crystal lattice is not modified. In fact, a slight local contraction of the crystal lattice of less than 5% accompanies the deprotonation, so that the morphology of the crystal and its spatial arrangement remain unchanged. Just some OH⁻ ions surrounding the iron cations may lose a proton H⁺ in situ, and become O²⁻ ions, leading correspondingly to the conversion of an Fe^{II} ion to Fe^{III} ion.

The invention in particular relates to the use of a LDH as defined above, in which the proportion of Fe^{II} replacing the divalent element is from about 1% (w/w) to 100% (w/w) relative to the total amount of divalent element.

In order to function, the LDH requires the presence of a minimum proportion of Fe^{II} of 1% permitting the conversion of an Fe^{II} ion to Fe^{III} ion. If the LDH does not contain Fe^{II}, the latter is then non-functional.

The invention relates more particularly to the use of a LDH as defined above, in which the proportion of Fe^{III} in the trivalent element is from 0% (w/w) to 100% (w/w) relative to the total amount of trivalent element.

The presence of Fe^{III} is not indispensable once Fe^{II} that is capable of being transformed to Fe^{III} is present in the LDH.

The invention also relates to the use of a LDH as defined above, in which M²⁺ is selected from Mg²⁺, Ni²⁺, Ca²⁺, Mn²⁺, and T³⁺ is selected from Al³⁺ and Cr³⁺.

The present invention relates more particularly to the use of a LDH as defined above, in the form of a ferrous-ferric oxyhydroxy salt as catalyst or as precursor of said catalyst, with the same crystalline structure as that of said catalyst, for the implementation of an oxidation-reduction process, said ferrous-ferric oxyhydroxy salt having the formula

[Fe^{II}₃ₙ₍₁₋ₓ₎Fe^{III}₃ₙₓO₆ₙHₙ₍₇₋₃ₓ₎]ⁿ⁺[Aⁿ⁻,mH₂O]ⁿ⁻,

in which Aⁿ⁻ is an anion with charge n, n having the values 1, 2 or 3, in particular 2, m is an integer varying from 1 to 10, in particular from 1 to 4, advantageously 3, and x is in the range from 0 to 1,

said ferrous-ferric oxyhydroxy salt being used in association with iron-reducing bacteria that are able to reduce Fe^{III} to Fe^{II} and in the presence of organic matter,

in order to reduce a substance S to a substance S_{reduced}, the redox potential of the pair S_{reduced}/S being greater than that of the pair Fe^{II}/Fe^{III} at the crystallographic sites of the Fe^{II}, x varying essentially in the range from 0.33 to 0.66, after the start-up of the oxidation-reduction process, without a substantial change in the crystalline structure of the aforesaid ferrous-ferric oxyhydroxy salt.

The term “catalyst” denotes here that the ferrous-ferric oxyhydroxy salt participates chemically in the oxidation-reduction process, and is regenerated during the process, as a result of the bacterial activity. By “catalyst” is meant a functional catalyst.

By “precursor with identical crystalline structure” is meant a ferrous-ferric oxyhydroxy salt with the same crystalline structure as that of the catalyst.

The catalyst has the formula [Fe^{II}_{3n (1-x)}Fe^{III}₃ₙₓO₆ₙH_{n (7-3x)}]ⁿ⁺[Aⁿ⁻, m H₂O]ⁿ⁻, in which x varies from 0.33 to 0.66 and the precursor has the same formula in which x can be less than 0.33 or greater than 0.66, and x can reach values from 0 to 1.

The start-up phase of the oxidation-reduction process permits the functional catalyst to be obtained from its precursor.

The inventors have shown that the ferrous-ferric oxyhydroxy salt as defined above can serve as a catalyst in oxidation-reduction processes in order to reduce a substance S and that the iron-reducing bacteria are effectively bacteria which thus make it possible to reduce nitrates indirectly.

The ferrous-ferric hydroxy salts belong to the class of lamellar double hydroxides, which have cationic lamellae comprising Fe^{II} and Fe^{III} ions of structure Fe(OH)₂, called brucite lamellae, and interlayers comprising anions and water molecules which counterbalance the excess of positive charges due to the Fe^{III} ions.

The ferrous-ferric oxyhydroxy salts have, for their part, a crystallographic structure similar to that of the hydroxy salts proper, but some of their OH⁻ ions surrounding each Fe^{III} cation are deprotonated while becoming O²⁻ ions. Fe^{II} ions oxidize to Fe^{III} to compensate the charge.

The ferrous-ferric oxyhydroxy salts used in the invention can be of natural origin or synthetic.

The ferrous-ferric oxyhydroxy salts observed in the natural state in soils only occur in a range of x between 0.33 and 0.66. It is the mineral fougerite. In contrast, the synthetic products correspond to values of x ranging from 0 to 1, owing to appropriate novel electronic properties.

The value of x within the ferrous-ferric oxyhydroxy salt corresponds to the ratio Fe^{III}/(Fe^{II}+Fe^{III}) and can be directly measured in situ by Mössbauer spectrometry.

The crystallographic structure of the ferrous-ferric oxyhydroxy salts, and more particularly that of the oxyhydroxycarbonate, was described in detail by Génin et al. (CR Geoscience, 2006; Solid State Sciences, 2006).

The expression “crystalline structure” denotes that said ferrous-ferric oxyhydroxy salt is in the form of a hexagonal-base prismatic solid with a regular, repeating structure, formed from an ordered stack of atoms, molecules or ions, according to the laws of periodicity of translation called Bravais lattice and the spatial distribution of which has been determined (Génin et al., 2006, Solid State Sciences).

The three ranges of x varying from 0 to 0.33, 0.33 to 0.66 and 0.66 to 1 have now been elucidated (Génin et al., 2006, Geoscience).

Thus, a value of x greater than 0.66 corresponds to a structure that would imply more energy than could be attained under natural conditions, whereas there is preferential formation of magnetite, from Fe₃O₄ to γ—Fe₂O₃, with a spinel structure.

For values of x less than 0.33, the crystallographic structure is metastable. This crystallographic structure is then obtained by voltammetric cycling.

Voltammetric cycling is a process in which the voltage on the solid is varied continuously and cyclically with a potentiometer.

The term “metastable” denotes a system that corresponds to a local energy minimum but where this minimum is not the lowest, leaving the term “stable” for the latter.

Transition of the solid from x=0.33 to x=1 occurs continuously by progressive oxidation under conditions of intensive oxidation such as is obtained with hydrogen peroxide H₂O₂. It is a phenomenon of deprotonation within the compound, during which some OH⁻ ions become O²⁻, correspondingly converting Fe^{II} ions to Fe^{III}.

In particular, transition of the solid from x=0.33 to x=1 is obtained by direct oxidation of the stoichiometric compound, the ferrous-ferric hydroxy salt (x=0.33) of formula [Fe^{II}₂ₙFe^{III}ₙ(OH)₆ₙ]ⁿ⁺ [Aⁿ⁻, m H₂O]ⁿ⁻, under conditions of intensive oxidation such as obtained with hydrogen peroxide H₂O₂.

The continuous deprotonation of ferrous-ferric oxyhydroxycarbonate was demonstrated for the first time by Ruby et al., 2006, Environ. Sci. Technol. No other known oxide (whether or not containing iron) possesses such a phenomenon of continuous deprotonation.

The substance S is in particular present in a liquid medium. Said liquid medium is laden with organic matter to a varying degree.

The ferrous-ferric oxyhydroxy salt according to the invention possesses oxidation-reduction properties that are completely novel.

The invention therefore relates to the oxidation-reduction pairs S_{reduced}/S and Fe^{II}/Fe^{III} within the catalyst.

When the anion is the carbonate and x varies from 0.33 to 0.66, the redox potential (or electrode potential) of the pair Fe^{II}/Fe^{III} at the crystallographic sites of the Fe^{II} varies from −0.21 to +0.11 V (standard hydrogen reference electrode), which corresponds to a chemical potential varying from −600 kJ mol⁻¹ to −582 kJ mol⁻.

The expression “redox potential at the crystallographic sites of the Fe^{II}” denotes the chemical potential at which Fe^{II} is fixed in solution if there is equilibrium between solid and solution. The crystallographic site of Fe^{II} is therefore also the site where the Fe^{II} is located in the solid.

The term anion denotes any ion with a negative charge. Within the scope of the present invention, the anion has 1, 2 or 3 negative charges, and in particular 2 negative charges (for example carbonate).

When x is equal to 0, the ferrous-ferric oxyhydroxy salt becomes simply the ferrous oxyhydroxy salt of formula [Fe^{II}₃ₙO₆ₙH₇ₙ]ⁿ⁺ [Aⁿ⁻, m H₂O]ⁿ⁻. Protonation then occurs, during which OH⁻ becomes H₂O.

In particular, when the anion has two negative charges, the ferrous oxyhydroxy salt becomes the ferrous oxyhydroxy salt of formula [Fe^{II}₆O₁₂H₁₄]²⁺ A²⁻.

By way of example, for the carbonates, the ferrous oxyhydroxy salt is the ferrous oxyhydroxycarbonate of formula [Fe^{II}₆O₁₂H₁₄]²⁺ [CO₃²⁻, 3 H₂O]²⁻.

When x is equal to 1, the ferrous-ferric oxyhydroxy salt becomes simply the ferric oxyhydroxy salt of formula [Fe^{III}₃ₙO₆ₙH₄ₙ]ⁿ⁺ [Aⁿ⁻, m H₂O]ⁿ⁻.

In particular, when the anion has two negative charges, the ferric oxyhydroxy salt becomes the ferric oxyhydroxy salt of formula [Fe^{III}₆O₁₂H₈]²⁺A²⁻.

By way of example, for the carbonates, the ferric oxyhydroxy salt is the ferric oxyhydroxycarbonate of formula [Fe^{III}₆O₁₂H₈]²⁺ [CO₃²⁻, 3 H₂O]₂₋.

When x is in the range from 0.33 to 0.66, the ferrous-ferric oxyhydroxy salt is the chemical compound homologue of the mineral called “fougerite” (IMA 2003-057), which was identified for the first time in hydromorphous soils in the Fougères national forest (Ile et Vilaine, France).

The moment when the ferrous-ferric oxyhydroxy salt, the iron-reducing bacteria and the organic matter, and optionally the substance S, are brought into contact is called the “moment of initiation of the process” or “initial moment”.

The expression “once the process is in operation” denotes the moment starting from which the ferrous-ferric oxyhydroxy salt corresponds to a value of x in the range 0.33 to 0.66. The catalyst is then ready to function.

The phase that begins at the moment of initiation of the process and ends at the moment starting from which the ferrous-ferric oxyhydroxy salt corresponds to a value of x less than or equal to 0.66 is called the phase of “process start-up” or phase of “activation of the catalyst”.

When x is less than 0.66 at the moment of initiation of the process, the phase of process start-up no longer exists.

The value of x varies in the course of the oxidation-reduction process, and in a novel manner; this variation of x takes place in situ, and does not involve any substantial change in the structure of the ferrous-ferric oxyhydroxy salt. In fact, bacterial reduction takes place without dissolution of the ferric precursor followed by reprecipitation of the oxyhydroxy salt.

The invention in particular relates to the use as defined above, for the implementation of a process in which the substance S is reduced to a substance S_{reduced} by oxidation of Fe^{II} to Fe^{III} and in which the organic matter is oxidized at the end of the reduction of Fe^{III} to Fe^{II} by the iron-reducing bacteria.

There is therefore consumption of organic matter in the course of the process according to the invention.

The invention in particular relates to the use as defined above, for the implementation of a process in which Fe^{II} is regenerated from Fe^{III} and vice versa, cybernetically.

The regeneration of Fe^{II} from Fe^{III} only takes place if the catalyst is in a functioning state, in particular due to the presence of the substance to be reduced S.

The expression “cybernetically” denotes in a “continuous cyclical” manner, so long as exhaustion of the substance S or of the organic matter does not occur, and/or so long as the iron-reducing bacteria remain active.

Thus, the value of x is constantly adjusted as a function of the relative quantity of active bacteria and the amount of substance to be reduced.

The invention in particular relates to the use as defined above, in which the transfer of electrons between Fe^{II} and Fe^{III} takes place reversibly in situ within said catalyst.

The expression “reversibly” denotes that the transfer of electrons takes place both in the direction Fe^{II} to Fe^{III} (release of an electron by Fe^{II}), due to the substance S, and in the direction Fe^{III} to Fe^{II} (capture of an electron by Fe^{III}), due to the bacteria according to the electronic semi-reaction: Fe^{III}+e− [symbol] Fe^{II}.

The expression “in situ” denotes that the transfer of charges, electrons and protons, takes place via the catalyst, without change in the crystalline structure of the catalyst, or diffusion of matter.

The transfer of electrons between Fe^{II} and Fe^{III} is accompanied by a transfer of protons between OH⁻ and O²⁻, which also takes place reversibly in situ in said functional catalyst.

According to an advantageous embodiment, the invention relates to the use as defined above, in which said oxidation-reduction process takes place under conditions of anoxia.

The expression “under conditions of anoxia” denotes in the substantial absence of oxygen, as is generally the case in a biological medium where no external supply of oxygen takes place.

Preferably, a subject of the invention is the use as defined above, in which x is greater than 0.66 at the initial moment before the start-up of the oxidation-reduction process.

According to a particularly advantageous embodiment, a subject of the invention is the use as defined above, in which x is equal to 1 at the initial moment before the start-up of the oxidation-reduction process.

A subject of the present invention is the use as defined above, in which the anion is selected from carbonate, chloride, sulphate, fluoride, iodide, oxalate, methanoate.

According to an advantageous embodiment, a subject of the invention is the use as defined above, in which the anion is the carbonate.

The invention in particular relates to the use as defined above, in which the substance S is selected from inorganic pollutants such as nitrate, selenate, chromate, arsenate or from organic pollutants and in particular plant protection products.

The term “inorganic pollutants” denotes any inorganic pollutant, in particular nitrate, selenate, chromate, arsenate.

The term “organic pollutants” denotes pollutants comprising carbon-containing matter, in particular certain plant protection products.

The term “plant protection products” or “insecticides” denotes acaricides, bactericides, fungicides, herbicides, nematicides, rodenticides, mole poisons, molluscicides, corvicides, fumigants.

A preferred use according to the invention is characterized in that the substance S is the nitrate NO₃⁻, the nitrate being reduced to dinitrogen N₂.

The invention relates to the use as defined above, in which the bacteria are facultative aerobic-anaerobic bacteria.

Anaerobic bacteria are bacteria that live in the substantial absence of oxygen. Among these bacteria, those that do not need a substituted external electron acceptor for respiration are the fermentative bacteria.

The anaerobic bacteria used in the invention are selected from the bacteria with obligate respiration.

Facultative aerobic bacteria are bacteria that can live in the presence or in the substantial absence of oxygen.

The invention in particular relates to the use as defined above, in which the bacteria are selected from the genera Shewanella putrefaciens, Geobacteru sp.

The bacteria of the genus Shewanella are facultative aerobic bacteria.

The bacteria of the genus Geobacter are obligate anaerobes.

A subject of the present invention is also the use as defined above, in which the ferrous-ferric oxyhydroxy salt is formed from a precursor with crystalline structure different from that of said ferrous-ferric oxyhydroxy salt, said precursor being a ferric oxyhydroxide such as ferrihydrite, lepidocrocite, goethite, in the presence of iron-reducing bacteria and anions.

Ferrihydrite, lepidocrocite and goethite were extensively described by Cornel and Schwertmann (Iron oxides, Wiley-VHC, 2nd edition). They are allotropic forms of ferric oxyhydroxide FeOOH: the ferrihydrite corresponds to δ′—FeOOH, the lepidocrocite to γ—FeOOH, and the goethite to α—FeOOH.

The name with the ending “ite” is the inorganic homologue of the chemical compound.

The term “precursor with crystalline structure different from that of said ferrous-ferric oxyhydroxy salt” denotes any compound, with crystalline structure different from that of said ferrous-ferric oxyhydroxy salt, starting from which the ferrous-ferric oxyhydroxy salt as defined above can form directly or indirectly (optionally involving the formation of an intermediate).

In particular, the precursor can either be a ferric oxyhydroxide, or a ferric oxyhydroxy salt.

The invention in particular relates to the use of a LDH as defined above, in association with a metal selected from Cu(II), Ag(I), Cd(II), Ni(II), Hg(II), Pb(II) and Mn(II), preferably Cu(II), in a proportion from about 2 to 20% (w/w) relative to the total Fe.

The metal and more particularly copper makes it possible to increase the kinetics of the reaction, which then takes place much more easily by deprotonation. If the LDH is not used in association with a metal, the reaction is much longer and takes place by dissolution and reprecipitation.

The invention also relates to the use of a LDH as defined above, in association with phosphate ions in a proportion of at least 1%.

The phosphate ions are adsorbed on the catalyst and provide stabilization of the LDH lamellae. In the case of fougerite, the phosphates adsorbed on the latter prevent its disproportionation to magnetite.

The phosphate can be added to the catalyst but can also be supplied by the environment, in particular from septic tanks or even polluted catchment waters.

Process for reducing a substance S to a substance S_{reduced} comprising:
introducing a LDH, as catalyst or precursor of said catalyst with the same crystalline structure as that of said catalyst, said LDH containing a divalent cation M²⁺ partially or completely substituted with Fe^{II}, and a trivalent cation T³⁺ optionally substituted with Fe^{III}, of the following general formula:

[M²⁺_{(z)}Fe^{II}_{(1-y-z)}T³⁺ₜFe^{III}_{(y-t)}O₂H₂]ⁿ⁺[(y/n)Aⁿ⁻,mH₂O]ⁿ⁻

in which:
¼<y<⅓, z<1−y and t<y, Aⁿ⁻ is an anion with charge n, n having the values 1, 2 or 3, in particular 2, m is an integer varying from 1 to 10, in particular from 1 to 4, advantageously 3,
and the ratio x=(y−t)/(1−z−t) can vary from 0 to 1,
   said LDH being used in association with iron-reducing bacteria able to reduce Fe^{III} to Fe^{II} and in the presence of organic matter,
   if x is greater than 0.66 at the initial moment, a start-up phase of the oxidation-reduction process corresponding to the reduction of Fe^{III} to Fe^{II} within said LDH by said iron-reducing bacteria, leading to a change in x to a value less than or equal to 0.66, in order to obtain said LDH in the form of a catalyst, without a substantial change in crystalline structure of said LDH,
   a phase of catalytic reduction of the substance S, added to the whole comprising the LDH, the bacteria and the organic matter, to a substance S_{reduced} by oxidation of the Fe^{II} to Fe^{III} within said LDH coupled to a stage of catalytic oxidation of the organic matter by reduction of the Fe^{III} to Fe^{II}, the redox potential of the pair S_{reduced}/S being greater than that of the pair Fe^{II}/Fe^{III} at the crystallographic sites of the Fe^{II}.

At the initial moment, the ferrous-ferric oxyhydroxy salt as defined above is put in the presence of said iron-reducing bacteria and organic matter.

The substance S is introduced with the ferrous-ferric oxyhydroxy salt at the initial moment, during activation of the catalyst or after the start-up of the process.

In particular, when the value of x is greater than 0.66 at the initial moment, it may be advantageous to add the substance S once x has reached a value less than 0.66, after the start-up of the oxidation-reduction process.

Regardless of the value of x at the initial moment, the iron-reducing bacteria oxidize the organic matter in the course of their respiration. The final electron acceptor of the bacterial respiratory chain is still the Fe^{III}, which is thus reduced to Fe^{II} actually within the ferrous-ferric oxyhydroxy salt. The reduction of Fe^{III} to Fe^{II} therefore induces a decrease in the value of x, to a value less than or equal to 0.66 actually within the ferrous-ferric oxyhydroxy salt, without substantial change in its crystalline structure.

If x is less than or equal to 0.66, reduction of substance S to a substance S_{reduced} by oxidation of the Fe^{II} to Fe^{III} actually within the catalyst takes place in addition to the bacterial respiration.

The catalytic reduction of the substance S to a substance S_{reduced} by oxidation of Fe^{II} to Fe^{III} within the ferrous-ferric oxyhydroxy salt is coupled to the regeneration of Fe^{III} to Fe^{II} by bacterial reduction in correlation with the catalytic oxidation of the organic matter (see FIG. 1).

Said reduction of the substance S is described as catalytic, as this reaction is coupled to the enzymatic catalytic oxidation of the organic matter by reduction of the Fe^{III} to Fe^{II}. The catalytic reduction of the substance S replaces a possible direct reduction of the substance S by certain bacteria, which use it for their respiration.

The ferrous-ferric oxyhydroxy salt facilitates or even makes possible the reduction of the substance S by the bacteria.

The Fe^{III} resulting from the reduction of the substance S then constantly regenerates the Fe^{II} via the bacterial respiration. Thus, the catalyst itself is also constantly regenerated.

From the thermodynamic standpoint, the reduction of substance S would be substantially decreased, or even non-existent, when the value of x exceeds 0.66.

The invention in particular relates to a process for reducing a substance S to a substance S_{reduced} as defined above, in which said LDH is in the form of a ferrous-ferric oxyhydroxy salt, comprising:
introducing a ferrous-ferric oxyhydroxy salt, as catalyst or precursor of said catalyst with the same crystalline structure as that of said catalyst, having the formula

[Fe^{II}₃ₙ₍₁₋ₓ₎Fe^{III}₃ₙₓO₆ₙHₙ₍₇₋₃ₓ₎ⁿ⁺[Aⁿ⁻,mH₂O]ⁿ⁻
in which Aⁿ⁻ is an anion with charge n, n having the values 1, 2 or 3, in particular 2, and x is in the range from 0 to 1 and m is an integer varying from 1 to 10, in particular from 1 to 4, advantageously 3, at the initial moment, with iron-reducing bacteria able to reduce Fe^{III} to Fe^{II} and organic matter,
if x is greater than 0.66 at the initial moment, a start-up phase of the oxidation-reduction process corresponding to the reduction of Fe^{III} to Fe^{II} within said ferrous-ferric oxyhydroxy salt by said iron-reducing bacteria, leading to a change in x to a value less than or equal to 0.66, in order to obtain said ferrous-ferric oxyhydroxy salt in the form of a catalyst without a substantial change in the crystalline structure of said ferrous-ferric oxyhydroxy salt,
a phase of catalytic reduction of the substance S, added to the whole comprising the ferrous-ferric oxyhydroxy salt, the bacteria and the organic matter, to a substance S_{reduced} by oxidation of Fe^{II} to Fe^{III} within the ferrous-ferric oxyhydroxy salt coupled to a stage of catalytic oxidation of the organic matter by reduction of the Fe^{III} to Fe^{II},
the redox potential of the pair S_{reduced}/S being greater than that of the pair Fe^{II}/Fe^{III} at the crystallographic sites of the Fe^{II}.

A more particular subject of the invention is a process making it possible to reduce a substance S to a substance S_{reduced} as defined above, in which said LDH is used in association with a metal selected from Cu(II), Ag(I), Cd(II), Ni(II), Hg(II), Pb(II) and Mn(II), preferably Cu(II), in a proportion from about 2 to 20% (w/w) relative to the total Fe.

A more particular subject of the invention is a process making it possible to reduce a substance S to a substance S_{reduced} as defined above, in which said LDH is used in association with phosphate ions in a proportion of at least 1%.

The invention also relates to a process making it possible to reduce a substance S to a substance S_{reduced} comprising:
introducing a LDH, as catalyst precursor, said LDH containing a divalent cation M²⁺ partially or completely substituted with Fe^{II}, and a trivalent cation T³⁺ optionally substituted with Fe^{III} of the following general formula:

[M²⁺_{(z)}Fe^{II}_{(1-y-z)}T³⁺ₜFe^{III}_{(y-t)}O₂H₂]ⁿ⁺[(y/n)Aⁿ⁻,mH₂O]ⁿ⁻
in which:
¼<y<⅓, z<1−y and t<y, Aⁿ⁻ is an anion with charge n, n having the values 1, 2 or 3, in particular 2, m is an integer varying from 1 to 10, in particular from 1 to 4, advantageously 3,
and the ratio x=(y−t)/(1−z−t) varies from 0 to 1,
   said LDH being used in association with iron-reducing bacteria able to reduce Fe^{II} to Fe^{II} and in the presence of organic matter,
if x is greater than 0.66 at the initial moment, a start-up phase of the oxidation-reduction process corresponding to the reduction of the Fe^{III} to Fe^{II} within said LDH by said iron-reducing bacteria, leading to a change in x to a value less than or equal to 0.66, in order to obtain said LDH in the form of a catalyst, without a substantial change in the crystalline structure of said LDH,
a phase of catalytic reduction of the substance S, added to the whole comprising the LDH, the bacteria and the organic matter, to a substance S_{reduced} by oxidation of the Fe^{II} to Fe^{II} within said LDH coupled to a stage of catalytic oxidation of the organic matter by reduction of the Fe^{III} to Fe^{II}, the redox potential of the pair S_{reduced}/S being greater than that of the pair Fe^{II}/Fe^{III} at the crystallographic sites of the Fe^{II}.

The catalytic reduction of the substance S to a substance S_{reduced} by oxidation of the Fe^{II} to Fe^{II} within the LDH is therefore coupled to the regeneration of Fe^{III} to Fe^{II} by bacterial reduction.

The invention also relates to a process making it possible to reduce a substance S to a substance S_{reduced}, as defined above, comprising:
introducing a ferrous-ferric oxyhydroxy salt, as catalyst, having the formula

[Fe^{II}₃ₙ₍₁₋ₓ₎Fe^{III}₃ₙₓO₆ₙHₙ₍₇₋₃ₓ₎]ⁿ⁺[Aⁿ⁻,mH₂O]ⁿ⁻
in which Aⁿ⁻ is an anion with charge n, n having the values 1, 2 or 3, in particular 2, m is an integer varying from 1 to 10, in particular from 1 to 4, advantageously 3 and x is from 0.33 to 0.66 at the initial moment, with iron-reducing bacteria able to reduce Fe^{III} to Fe^{II} and organic matter,
a phase of catalytic reduction of substance S, added to the whole comprising the ferrous-ferric oxyhydroxy salt, the bacteria and the organic matter, to a substance S_{reduced} by oxidation of the Fe^{II} to Fe^{III} coupled to a stage of catalytic oxidation of the organic matter by reduction of the Fe^{III} to Fe^{II},
the redox potential of the pair S_{reduced}/S being greater than that of the pair Fe^{II}/Fe^{III} at the crystallographic sites of the Fe^{II}.

The catalytic reduction of the substance S to a substance S_{reduced} by oxidation of the Fe^{II} to Fe^{III} within the ferrous-ferric oxyhydroxy salt is therefore coupled to regeneration of the Fe^{III} to Fe^{II} by bacterial reduction.

The invention also relates to a process making it possible to reduce a substance S to a substance S_{reduced} comprising:
introducing a ferrous-ferric oxyhydroxy salt, as catalyst precursor, having the formula

[Fe^{II}₃ₙ₍₁₋ₓ₎Fe^{III}₃ₙₓO₆ₙHₙ₍₇₋₃ₓ₎]ⁿ⁺[Aⁿ⁻,mH₂O]ⁿ⁻

in which Aⁿ⁻ is an anion with charge n, n having the values 1, 2 or 3, in particular 2, m is an integer varying from 1 to 10, in particular from 1 to 4, advantageously 3 and x is greater than 0.66 at the initial moment,

with iron-reducing bacteria able to reduce the Fe^{III} to Fe^{II} and organic matter,
a start-up phase of the oxidation-reduction process corresponding to the reduction of Fe^{III} to Fe^{II} within said ferrous-ferric oxyhydroxy salt by said iron-reducing bacteria, leading to a change in x to a value less than or equal to 0.66, in order to obtain said ferrous-ferric oxyhydroxy salt in the form of a catalyst without a substantial change in the crystalline structure of said ferrous-ferric oxyhydroxy salt,
a phase of catalytic reduction of the substance S, added to the whole comprising the ferrous-ferric oxyhydroxy salt, the bacteria and the organic matter, to a substance S_{reduced} by oxidation of the Fe^{II} to Fe^{III} within said ferrous-ferric oxyhydroxy salt coupled to a stage of catalytic oxidation of the organic matter by reduction of the Fe^{III} to Fe^{II}
the redox potential of the pair S_{reduced}/S being greater than that of the pair Fe^{II}/Fe^{III} at the crystallographic sites of the Fe^{II}.
The catalytic reduction of substance S to a substance S_{reduced} by oxidation of the

Fe^{II} to Fe^{III} within the ferrous-ferric oxyhydroxy salt is therefore coupled to the regeneration of Fe^{III} to Fe^{II} by bacterial reduction.

The invention in particular relates to a process for reducing a substance S to a substance S_{reduced}, as defined above, in which said LDH is used in association with a metal selected from Cu(II), Ag(I), Cd(II), Ni(II), Hg(II), Pb(II) and Mn(II), preferably Cu(II), in a proportion from about 2 to 20% (w/w) relative to the total Fe.

A more particular subject of the invention is a process making it possible to reduce a substance S to a substance S_{reduced}, as defined above, in which said LDH is used in association with phosphate ions in a proportion of at least 1%.

The invention in particular relates to a process as defined above, in which x is equal to 1 at the initial moment.

A subject of the invention is a process as defined above, said process taking place under conditions of anoxia.

The conditions of anoxia are in particular obtained in a confined medium leading to a substantial absence of oxygen and can be obtained as a result of the bacterial activity.

The invention in particular relates to a process as defined above, in which Fe^{II} is regenerated from Fe^{III} and vice versa, cybernetically.

The invention in particular relates to a process as defined above, in which the transfer of electrons between Fe^{II} and Fe^{III} takes place reversibly in situ in said catalyst.

The invention in particular relates to a process as defined above, in which the anion is selected from carbonate, chloride, sulphate, fluoride, iodide, oxalate, methanoate.

According to a preferred embodiment, the invention relates to the process as defined above in which the anion is the carbonate and said ferrous-ferric oxyhydroxy salt is a ferrous-ferric oxyhydroxycarbonate of formula

[Fe^{II}₆₍₁₋ₓ₎Fe^{III}₆ₓO₁₂H₂₍₇₋₃ₓ₎]²⁺[CO₃²⁻,3H₂O]²⁻.

According to an advantageous embodiment of the process as defined above, the substance S is selected from inorganic pollutants such as nitrate, selenate, chromate, arsenate or from organic pollutants, in particular plant protection products.

According to a particularly preferred embodiment of the process according to the invention, the substance S is the nitrate NO₃⁻, the nitrate being reduced to dinitrogen N₂ (see FIG. 3).

A subject of the invention is the process as defined above, in which the bacteria are facultative aerobic-anaerobic bacteria.

The invention in particular relates to the process as defined above, in which the bacteria are selected from Shewanella putrefaciens, Geobacter sp.

In an advantageous embodiment of the process according to the invention, the ferrous-ferric oxyhydroxy salt is formed from a precursor with a crystalline structure different from that of said ferrous-ferric oxyhydroxy salt, said precursor being a ferric oxyhydroxide such as ferrihydrite, lepidocrocite, goethite, in the presence of iron-reducing bacteria and anions.

The invention relates to a process as defined above, characterized in that the pH is in the range from 5 to 10, and in particular 7.

When the pH is less than 5 or greater than 10, the activity of the catalyst and the activity of the bacteria may be diminished.

The invention also relates to a process as defined above, characterized in that the temperature varies from 5° C. to 30° C.

With temperatures above 30° C. there is a risk of promoting the formation of magnetite mixed with siderite FeCO₃ from the ferrous-ferric oxyhydroxy salt.

Temperatures below 5° C. slow down the kinetics of the oxidation-reduction reactions.

The process according to the invention operates in particular in conditions of temperature and pH that are encountered in particular in a temperate climate, for example in natural hydromorphous soils.

A hydromorphous soil is a waterlogged soil whose morphology is due to the presence of water. For example, the soil of an aquifer or the soils of river valleys are hydromorphous. The great majority of soils in temperate zones are hydromorphous, to a varying depth from a metre to 100 metres.

The ferrous-ferric oxyhydroxy salt used in the process according to the invention can be obtained by chemical synthesis or by bacterial synthesis.

The present invention in particular relates to a process as defined above, in which the ferrous-ferric oxyhydroxy salt is obtained by oxidation of a precipitate of Fe(OH)₂ in the presence of anions, comprising:
a stage of preparation of a precipitate of Fe(OH)₂, in particular by mixing in solution a ferrous salt [Fe^{II}] A²⁻ with a base, in particular NaOH
a stage of stirring of said mixture in the presence of air, in order to obtain a ferrous-ferric hydroxy salt of formula

[Fe^{II}₍₁₋ₓ₎Fe^{III}ₓ(OH)₂]^{x+}[(x/n)Aⁿ⁻]^{x-}
in which x varies in the range from 0.25 to 0.33,
a stage of deprotonation by oxidation with H₂O₂ or pure O₂ in solution or by oxidation in the open air after drying,
in order to obtain a ferrous-ferric oxyhydroxy salt in which x is greater than 0.33.

In the stage of preparation of the precipitate of Fe(OH)₂, the concentration of the base is advantageously equivalent to 5/3 of the concentration of Fe^{II} (Génin et al., 2006, Geoscience).

Advantageously, a ferrous-ferric oxyhydroxy salt of formula [Fe^{II}₂ₙFe^{III}ₙ(OH)₆ₙ]ⁿ⁺ [Aⁿ⁻, m H₂O]ⁿ⁻ corresponding to x=0.33) is then prepared first by introducing ⅔ of Fe^{II} and ⅓ of Fe^{III}. Secondly, H₂O₂ is added in stoichiometric proportions to obtain a ferrous-ferric oxyhydroxy salt corresponding to the desired value of x, greater than 0.33, according to the deprotonation reaction.

This deprotonation reaction of the ferrous-ferric hydroxy salt by H₂O₂ is as follows:

[Fe^{II}₂ₙFe^{III}ₙ(OH)₆ₙ]ⁿ⁺Aⁿ⁻+(3x−1)H₂O₂→[Fe^{II}₃ₙ₍₁₋ₓFe^{III}₃ₙₓO₆ₙHₙ₍₇₋₃ₓ₎]ⁿ⁺Aⁿ⁻⁺ⁿ⁽3x−1)H₂O,

Heretofore and hereinafter, the deprotonation of the ferrous-ferric oxyhydroxy salt can be obtained by the addition of pure O₂ instead of H₂O₂.

The present invention also relates to a process as defined above in which the ferrous-ferric oxyhydroxy salt is prepared by co-precipitation of the Fe^{II} and Fe^{III} ions in the presence of anions, comprising the following stages:
preparation of a solution of Fe^{II}, Fe^{III} and anions, the ratio [concentration of Fe^{III}]/[concentration of Fe^{II} and Fe^{III}] being equal to x,
addition of a solution of a base, in particular NaOH, to said solution of Fe^{II} and Fe^{III} in the absence of oxygen, to obtain a ferrous-ferric hydroxy salt of formula

[Fe^{II}₍₁₋ₓ₎Fe^{III}ₓ(OH)₂]^{x+}[(x/n)Aⁿ⁻]^{x-}
in which x varies in the range from 0.25 to 0.33,
a stage of deprotonation by oxidation with H₂O₂ or pure O₂ in solution or by oxidation in the open air after drying,
in order to obtain a ferrous-ferric oxyhydroxy salt in which x is greater than 0.33.

The deprotonation reaction of the ferrous-ferric hydroxy salt is as follows:

[Fe^{II}₂ₙFe^{III}ₙ(OH)₆ₙ]ⁿ⁺Aⁿ⁻+(3x−1)H₂O₂→[Fe^{II}₃ₙ₍₁₋ₓFe^{III}₃ₙₓO₆ₙHₙ₍₇₋₃ₓ₎]ⁿ⁺Aⁿ⁻+n(3x−1)H₂O,

In the first stage of preparation of the solution of Fe^{II}, Fe^{III} and anions, the total iron concentration in said solution is typically comprised between 0.1 and 2 M, in particular 0.4 M and the concentration of anions is greater than stoichiometric (Ruby et al., 2006, Geoscience).

Addition of the base is preferably carried out at ambient temperature.

A subject of the present invention is also a process as defined above, in which the ferrous-ferric oxyhydroxy salt is prepared by bacterial synthesis, comprising: culture of iron-reducing bacteria under conditions of anoxia in a suitable medium comprising:
Fe^{III}, in particular in the form of an oxyhydroxide or of a ferric oxyhydroxy salt of formula [Fe^{III}₃ₙO₆ₙH₄ₙ]ⁿ⁺ [Aⁿ⁻, m H₂O]⁻,
organic matter, in particular the methanoate HCO₂⁻, and
an anion Aⁿ⁻, if the anion is not HCO₃⁻,
   in order to obtain a ferrous-ferric oxyhydroxy salt in which x varies from 0.33 to 0.66.

In the culture stage, the Fe^{III} present in the medium is in particular in the form of an oxyhydroxide FeOOH or of a ferric oxyhydroxy salt of formula

[Fe^{III}₃ₙO₆ₙH₄ₙ]ⁿ⁺[Aⁿ⁻,mH₂O]ⁿ⁻.

Advantageously, said suitable medium for the culture of iron-reducing bacteria includes Fe^{III} at a concentration ranging from 20 mM to 200 mM, in particular 80 mM.

When the organic matter used is methanoate, an optimum concentration in said suitable medium is in the range from 5 mM to 200 mM, in particular from 20 to 75 mM.

Optionally, anthraquinone-2,6-disulphonate at a concentration from about 200 μM to about 500 μM, in particular 100 μM, can be added to said culture medium.

The incubation stage is carried out under conditions of temperature and stiffing appropriate to the strain of bacteria used.

The temperature is in particular comprised in the range from 10° C. to 40° C., and incubation is preferably carried out with stirring.

The ferrous-ferric oxyhydroxy salt obtained at the end of the culture stage is preferably dried, in particular by pumping under vacuum.

According to an advantageous embodiment, the invention relates to a process as defined above, for the pollution control of a medium to be treated.

The medium to be treated is a liquid medium, which can be a medium laden with organic matter to a varying extent.

The organic burden of the medium to be treated can be very high, for example in a medium of the sludge type.

The media to be treated to which the present invention relates in particular are as follows: spring water or well water or catchment water from aquifers, water from run-off, watercourses, ponds, lakes, wells; municipal, industrial and agricultural wastewater, in particular individual sanitation, water for distribution networks and water from treatment works, water from individual and semi-collective sanitation (septic tanks).

The media to be treated can in particular be water in aquifers and watercourses. As an example, the nitrate content of the latter in Brittany is close to or even frequently exceeds the current legal limit of potability of 50 mg/l. The European Commission wishes to halve this limit irreversibly by 2013.

The process as defined above can also be used in sanitation of scattered settlements, for water treatment supplementary to that of septic tanks at their outlet, but also in agriculture, for example in order to lower the nitrate level in liquid manure before spreading.

Thus, it is envisaged to measure continuously, by means of sensors, the nitrate content in a liquid manure pit, so as to carry out spreading when said nitrate content has reached the desired value. These are processes that are relatively easy to implement and maintain, and do not require conditions of anoxia in a compartment containing the catalyst.

The invention also relates to the use of a process as defined above, for limiting the excessive proliferation of algae, in particular ulvae.

The algae are in particular marine algae.

The excessive proliferation of algae arises in particular from the presence of certain pollutants, such as phosphates and nitrates.

The excessive proliferation of algae of the ulva type results in particular from pollution with nitrates, which are discharged excessively into the sea and which determine the proliferation of said algae. This is so, for example, in the case of environmental conditions such as are often observed in Brittany.

In an advantageous embodiment, the process according to the invention is applied to the water of a drainage basin, in order to reduce the amount of pollutants, in particular nitrates, in water that is discharged into the sea.

The invention also relates to a product comprising at least one LDH, said LDH containing a divalent cation M²⁺ partially or completely substituted with Fe^{II}, and a trivalent cation T³⁺ optionally substituted with Fe^{III}, of the following general formula:

[M²⁺_{(z)}Fe^{II}_{(1-y-z)}T³⁺ₜFe^{III}_{(y-t)}O₂H₂]ⁿ⁺[(y/n)Aⁿ⁻,mH₂O]ⁿ⁻
in which:
¼<y<⅓, z<1−y and t<y, A' is an anion with charge n, n having the values 1, 2 or 3, in particular 2, m is an integer varying from 1 to 10,
in particular from 1 to 4, advantageously 3,
and the ratio x=(y−t)/(1−z−t), which can vary from 0 to 1, is in particular 1, in crystalline form, the ratio of surface volume to specific volume being greater than 100, without a substantial change in the crystalline structure of said LDH.

The LDH as defined above is a catalyst or a precursor of said catalyst.

The LDH as defined above can in particular have a granulometry in the nanometre range.

Regardless of the grain size of the LDH according to the invention, the redox potential of the Fe^{III}/Fe^{II} pair within said LDH remains the same.

With an increase in the ratio of surface volume to specific volume, the accessibility of the pollutant to the catalyst increases, and therefore the catalyst is more effective.

The LDH does not undergo a substantial change in its crystalline structure, or its morphology.

The invention in particular relates to a product as defined above, in which said LDH is constituted by at least one ferrous-ferric oxyhydroxy salt having the formula:

[Fe^{II}₃ₙ₍₁₋ₓ₎Fe^{III}₃ₙₓO₆ₙHₙ₍₇₋₃ₓ₎]ⁿ⁺[Aⁿ⁻,mH₂O]ⁿ⁻

in which Aⁿ⁻ is an anion with charge n, n having the values 1, 2 or 3, in particular 2, m is an integer varying from 1 to 10, in particular from 1 to 4, advantageously 3 and x is in the range from 0 to 1, in crystalline form, the ratio of surface volume to specific volume being greater than 100, without a substantial change in the crystalline structure of said ferrous-ferric oxyhydroxy salt.

The ferrous-ferric oxyhydroxy salt as defined above is a catalyst or a precursor of said catalyst.

The ferrous-ferric oxyhydroxy salt as defined above can in particular have a granulometry in the nanometre range.

Regardless of the grain size of the ferrous-ferric oxyhydroxy salt according to the invention, the redox potential of the Fe^{III}/Fe^{II} pair within said ferrous-ferric oxyhydroxy salt remains the same.

With an increase in the ratio of surface volume to specific volume, the accessibility of the pollutant to the catalyst increases, and therefore the catalyst is more effective.

The ferrous-ferric oxyhydroxy salt does not undergo a substantial change in its crystalline structure, or its morphology.

According to an advantageous embodiment, the product defined above is the ferrous-ferric oxyhydroxycarbonate of formula:

[Fe^{II}₆₍₁₋ₓ₎Fe^{III}₆ₓO₁₂H₂₍₇₋₃ₓ₎]²⁺[CO₃²⁻,3H₂O]²⁻

in which the anion is the carbonate and x is between 0 and 1.

According to a particularly advantageous embodiment, the product defined above is the ferric oxyhydroxycarbonate of formula:

[Fe^{III}₆O₁₂H₈]²⁺[CO₃²⁻,3H₂O]²⁻.

The invention in particular relates to a product as defined above, in which said LDH is used in association with a metal selected from Cu(II), Ag(I), Cd(II), Ni(II), Hg(II), Pb(II) and Mn(II), preferably Cu(II), in a proportion from about 2 to 20% (w/w) relative to the total Fe.

The invention in particular relates to a product as defined above, in which said LDH is used in association with phosphate ions in a proportion of at least 1%.

The invention also relates to a product constituted by at least one support coated with at least one LDH, said LDH containing a divalent cation M²⁺ partially or completely substituted with Fe^{II}, and a trivalent cation T³⁺ optionally substituted with Fe^{III}, of the following general formula:

[M²⁺_{(z)}Fe^{II}_{(1-y-z)}T³⁺ₜFe^{III}_{(y-t)}O₂H₂]ⁿ⁺[(y/n)Aⁿ⁻,mH₂O]ⁿ⁻
in which:
¼<y<⅓, z<1−y and t<y, Aⁿ⁻ is an anion with charge n, n having the values 1, 2 or 3, in particular 2, m is an integer varying from 1 to 10,
in particular from 1 to 4, advantageously 3,
and the ratio x=(y−t)/(1−z−t) varies from 0 to 1, in particular 1,
in crystalline form, the support in particular being selected from sand, clay, polymer beads.

The invention relates to a product as defined above, characterized in that the support is selected from sand, clay, polymer beads.

A product that is preferred according to the invention is characterized in that the support has a granulometry from about 50 μm to about 200 μm, in particular of about 100 μm.

The invention in particular relates to a product as defined above, in which said LDH is constituted by at least one ferrous-ferric oxyhydroxy salt having the formula:

[Fe^{II}₃ₙ₍₁₋ₓ₎Fe^{III}₃ₙₓO₆ₙHₙ₍₇₋₃ₓ₎]ⁿ⁺[Aⁿ⁻,mH₂O]ⁿ⁻

in which Aⁿ⁻ is an anion with charge n, n having the values 1, 2 or 3, in particular 2, m is an integer varying from 1 to 10, in particular from 1 to 4, advantageously 3, and x is in the range from 0 to 1, in crystalline form.

The ferrous-ferric oxyhydroxy salt can be obtained by chemical synthesis as defined above or by bacterial reduction of ferric oxyhydroxides such as ferrihydrite, lepidocrocite or goethite.

An advantageous product according to the invention is a product as defined above constituted by at least one support coated with at least one ferrous-ferric oxyhydroxycarbonate having the formula:

[Fe^{II}₆₍₁₋ₓ₎Fe^{III}₆ₓO₁₂H₂₍₇₋₃ₓ₎]²⁺[CO₃²⁻,3H₂O]²⁻

in which the anion is the carbonate and x is comprised from 0 to 1, in crystalline form.

Preferably, the product as defined is constituted by at least one support coated with a ferrous-ferric oxyhydroxycarbonate.

In a preferred embodiment of the invention, the product as defined above is characterized in that x is equal to 1.

In particular, it is the ferric oxyhydroxycarbonate of formula [Fe^{III}₆O₁₂H₈]²⁺[CO₃²⁻, 3 H₂O]²⁻.

The invention in particular relates to a product as defined above, in which said LDH is used in association with a metal selected from Cu(II), Ag(I), Cd(II), Ni(II), Hg(II), Pb(II) and Mn(II), preferably Cu(II), in a proportion from about 2 to 20% (w/w) relative to the total Fe.

A more particular subject of the invention is a product as defined above, in which said LDH is used in association with phosphate ions in a proportion of at least 1%.

A product particularly preferred according to the invention is characterized in that the ratio of the volume of surface deposit of ferrous-ferric oxyhydroxy salt to the volume of support is between about 1/100 and about 1/10000, in particular 1/1000.

The products as defined above can be obtained by the implementation of the following operations: “dry” preparation of the coating that is deposited on a support, preparation of the coating “in solution”, or preparation of the coating, in the course of which the support is added at the very moment of synthesis of the ferrous-ferric oxyhydroxy salt.

When the product comprises ferrous-ferric oxyhydroxy salt of formula:

[Fe^{II}₃ₙ₍₁₋ₓ₎Fe^{III}₃ₙₓO₆ₙHₙ₍₇₋₃ₓ₎]ⁿ⁺[Aⁿ⁻,mH₂O]ⁿ⁻

in which Aⁿ⁻ is an anion with charge n, n having the values 1, 2 or 3, in particular 2, m is an integer varying from 1 to 10, in particular from 1 to 4, advantageously 3, and x is different from 1, preparation of the product is carried out under conditions of anoxia to avoid oxidation of said ferrous-ferric oxyhydroxy salt.

In a preferred embodiment, the product as defined above comprises a ferric oxyhydroxy salt of formula [Fe^{III}₃ₙO₆ₙH₄ₙ]ⁿ⁺[Aⁿ⁻, m H₂O]ⁿ⁻, Aⁿ⁻ being an anion with charge n, n having the values 1, 2 or 3, in particular 2 and m is an integer varying from 1 to 10, in particular from 1 to 4, advantageously 3.

The invention in particular relates to a support coated with a ferric oxyhydroxy salt, as defined above, of formula [Fe^{III}₃ₙO₆ₙH₄ₙ]ⁿ⁺ [Aⁿ⁻, m H₂O]ⁿ⁻, in which Aⁿ⁻ is an anion with charge n, n having the values 1, 2 or 3, in particular 2, and m is an integer varying from 1 to 10, in particular from 1 to 4, advantageously 3, as obtained by the implementation of the process comprising the stages of:
coprecipitation in solution of Fe^{II} and Fe^{III} ions in the presence of anions Aⁿ⁻ in the absence of oxygen, to obtain a ferrous-ferric hydroxy salt of formula

[Fe^{II}₂ₙFe^{III}ₙ(OH)₆ₙ]ⁿ⁺[Aⁿ⁻, m H₂O]ⁿ⁻,
complete and rapid oxidation, by H₂O₂ or pure O₂ in solution or in air of said dry ferrous-ferric hydroxy salt after drying, in order to obtain a ferric oxyhydroxy salt of formula

[Fe^{III}₃ₙO₆ₙH₄ₙ]ⁿ⁺[Aⁿ⁻,mH₂O]ⁿ⁻,
drying of said ferric oxyhydroxy salt, in order to obtain a dry ferric oxyhydroxy salt, and
mixing the dry ferric oxyhydroxy salt with said support, in order to obtain said support coated with a ferric oxyhydroxy salt.

Drying, for example under vacuum, in particular makes it possible to obtain a product constituted by less than 1% of water by weight.

The mixing stage is in particular carried out mechanically.

Advantageously, during mixing, the ferric oxyhydroxy salt is in excess relative to the support.

After the mixing stage, the support coated with the ferric oxyhydroxy salt can be washed, in particular with distilled water.

The invention also relates to a support coated with a ferric oxyhydroxy salt of formula Fe^{III}₃ₙO₆ₙH₄ₙ [Aⁿ⁻, m H₂O]ⁿ⁻, in which Aⁿ⁻ is an anion with charge n, and m is an integer varying from 1 to 10, in particular from 1 to 4, advantageously 3, as obtained by implementation of the process comprising the stages of:
coprecipitation in solution of Fe^{II} and Fe^{III} ions in the presence of anions Aⁿ⁻ in the absence of oxygen, in order to obtain a ferrous-ferric hydroxy salt of formula

[Fe^{II}₂ₙFe^{III}ₙ(OH)₆ₙ]ⁿ⁺[Aⁿ⁻,mH₂O]ⁿ⁻
in which Aⁿ⁻ is an anion with charge n,
complete and rapid oxidation, by H₂O₂ or pure O₂ in the solution of said ferrous-ferric hydroxy salt, in order to obtain a ferric oxyhydroxy salt of formula

[Fe^{III}₃ₙO₆ₙH₄ₙ]ⁿ⁺[Aⁿ⁻,mH₂O]ⁿ⁻
addition of said support to said solution, in order to obtain a support coated with the ferric oxyhydroxy salt in solution, and
filtration and drying of said support coated with the ferric oxyhydroxy salt in solution, in order to obtain said support coated with a ferric oxyhydroxy salt.

The invention also relates to a support coated with a ferric oxyhydroxy salt of formula [Fe^{III}₃ₙO₆ₙH₄ₙ]ⁿ⁺ [Aⁿ⁻, m H₂O]ⁿ⁻, in which Aⁿ⁻ is an anion with charge n, and m is an integer varying from 1 to 10, in particular from 1 to 4, advantageously 3, as obtained by implementation of the process comprising:
introducing Fe^{II} and Fe^{III} ions, anions Aⁿ⁻, H₂O₂ or O₂ and support in solution, and
coprecipitation of said Fe^{II} and Fe^{III} ions in the presence of anions Aⁿ⁻ and immediate simultaneous oxidation by H₂O₂ or O₂, in order to obtain said support coated with the ferric oxyhydroxy salt of formula

[Fe^{III}₃ₙO₆ₙH₄ₙ]ⁿ⁺[Aⁿ⁻,mH₂O]ⁿ⁻.

In a particular embodiment, the invention relates to a support coated with a ferric oxyhydroxycarbonate of formula [Fe^{III}₆O₁₂H₈]²⁺ [CO₃²⁻, 3 H₂O]²⁻, as obtained by implementation of the process comprising the stages of:
coprecipitation in solution of Fe^{II} and Fe^{III} ions in the presence of carbonate anions in the absence of oxygen, in order to obtain a ferrous-ferric hydroxycarbonate of formula

[Fe^{II}₄Fe^{III}₂(OH)₁₂]²⁺[CO₃²⁻,3H₂O]²⁻,
complete and rapid oxidation, by adding H₂O₂ or pure O₂ to the solution or in air, of said dry ferrous-ferric hydroxycarbonate after drying, in order to obtain a ferric oxyhydroxycarbonate of formula

[Fe^{III}₆O₁₂H₈]²⁺[CO₃²⁻,3H₂O]²⁻,
drying of said ferric oxyhydroxycarbonate, in order to obtain a dry ferric oxyhydroxycarbonate, and
mixing of the dry ferric oxyhydroxycarbonate with said support, to obtain said support coated with the ferric oxyhydroxycarbonate.

After the mixing stage, the support coated with the ferric oxyhydroxy salt can be washed, in particular with distilled water.

In a particular embodiment, the invention relates to a support coated with the ferric oxyhydroxycarbonate of formula [Fe^{III}₆O₁₂H₈]²⁺[CO₃²⁻, 3 H₂O]²⁻, as obtained by implementation of the process comprising the stages of:
coprecipitation in solution of Fe^{II} and Fe^{III} ions in the presence of carbonate anions in the absence of oxygen, in order to obtain a ferrous-ferric hydroxycarbonate of formula

[Fe^{II}₄Fe^{III}₂(OH)₁₂]²⁺[CO₃²⁻,3H₂O]²⁻,
complete and rapid oxidation, by H₂O₂ or pure O₂ in the solution of said ferrous-ferric hydroxycarbonate, in order to obtain a ferric oxyhydroxycarbonate of formula

[Fe^{III}₆O₁₂H₈]²⁺[CO₃²⁻,3H₂O]²⁻,
addition of said support to said solution, in order to obtain a support coated with the ferric oxyhydroxycarbonate in solution, and
filtration and drying of said support coated with the ferric oxyhydroxycarbonate in solution, in order to obtain said support coated with ferric oxyhydroxycarbonate.

In a particular embodiment, the invention relates to a support coated with ferric oxyhydroxycarbonate of formula [Fe^{III}₆O₁₂H₈]²⁺ [CO₃², 3H₂O]²⁻, as obtained by implementation of the process comprising:
introducing Fe^{II} and Fe^{III} ions, carbonate anions, H₂O₂ or O₂ and said support in solution, and
coprecipitation of said Fe^{II} and Fe^{III} ions in the presence of carbonate anions and immediate simultaneous oxidation by H₂O₂ or pure O₂, in order to obtain said support coated with the ferric oxyhydroxycarbonate of formula [Fe^{III}₆O₁₂H₈]²⁺[CO₃²⁻, 3 H₂O]²⁻.

The present invention also relates to a kit comprising:

at least one LDH, said LDH containing a divalent cation M²⁺ partially or completely substituted with Fe^{II}, and a trivalent cation T³⁺ optionally substituted with Fe^{III}, of the following general formula:

[M²⁺_{(z)}Fe^{II}_{(1-y-z)}T³⁺ₜFe^{III}_{(y-t)}O₂H₂]ⁿ⁺[(y/n)Aⁿ⁻,mH₂O]ⁿ⁻
in which:
¼<y<⅓, z<1−y and t<y, A' is an anion with charge n, n having the values 1, 2 or 3, in particular 2, m is an integer varying from 1 to 10,
in particular from 1 to 4, advantageously 3,
and the ratio x=(y−t)/(1−z−t) which can vary from 0 to 1, in particular 1, in crystalline form,
   at least one support, in particular selected from sand, clay, polymer beads,
to be used simultaneously, separately or spread over time, intended for the implementation of a process of pollution control of a medium to be treated.

The present invention in particular relates to a kit as defined above in which said LDH is a ferrous-ferric oxyhydroxy salt, comprising:
at least one ferrous-ferric oxyhydroxy salt having the formula:

[Fe^{II}₃ₙ₍₁₋ₓ₎Fe^{III}₃ₙₓO₆ₙHₙ₍₇₋₃ₓ₎]ⁿ⁺[Aⁿ⁻,mH₂O]ⁿ⁻

in which Aⁿ⁻ is an anion with charge n, n having the values 1, 2 or 3, in particular 2, m is an integer varying from 1 to 10, in particular from 1 to 4, advantageously 3 and x is in the range from 0 to 1, in crystalline form,

at least one support, in particular selected from sand, clay, polymer beads, to be used simultaneously, separately or spread over time, intended for the implementation of a process of pollution control of a medium to be treated.

In a preferred kit according to the invention, the ferrous-ferric oxyhydroxy salt is the ferrous-ferric oxyhydroxycarbonate of formula:

[Fe^{II}₆₍₁₋ₓ₎Fe^{III}₆ₓO₁₂H₂₍₇₋₃ₓ₎]²⁺[CO₃²⁻,3H₂O]²⁻.

The invention in particular relates to a kit as defined above in which the ferrous-ferric oxyhydroxy salt is a ferric oxyhydroxy salt of formula:

[Fe^{III}₃ₙO₆ₙH₄ₙ]ⁿ⁺[Aⁿ⁻,mH₂O]ⁿ⁻.

It is the fully oxidized form of the ferrous-ferric oxyhydroxy salt.

In another preferred kit according to the invention, the ferrous-ferric oxyhydroxy salt is a ferric oxyhydroxycarbonate of formula:

[Fe^{III}₆O₁₂H₈]²⁺[CO₃²⁻,3H₂O]²⁻.

The invention also relates to the use of a product as defined above or of a kit as defined above, for the implementation of a process for the catalytic reduction of a substance S to a substance S_{reduced}, the redox potential of the pair S_{reduced}/S being greater than that of the pair Fe^{III}/Fe^{II} at the crystallographic sites of the Fe^{II}.

The invention in particular relates to the use of a product as defined above or of a kit as defined above, for the implementation of a process of pollution control of a medium to be treated.

In a preferred embodiment, the invention relates to the use of a product as defined above or of a kit as defined above, for limiting the excessive proliferation of algae, in particular ulvae.

The invention can be applied within the scope of a regional development, for the general improvement of the quality of the waters in the environment and to create developed zones where these arrangements would make it possible to improve the natural conditions of denitrification. These forms of development are related to the techniques of infiltration/percolation in waterlogged areas to be treated, which could be combined with lagooning and are called hereinafter “Waterlogged Areas Reinforced by Iron Purification (WARIP)”.

The starting materials used for the ferric species can realistically no longer be synthetic ferric oxyhydroxycarbonate as in the case of the previous reactors, given the amount of product required. The starting materials used are therefore natural ferric oxyhydroxides, as found in iron ores, to which organic matter is added in the form of compost. The natural community of bacteria present in compost is sufficient to initiate the oxidation-reduction reactions.

Inert minerals can be added in order to increase the state of division of the ferric oxyhydroxides, to serve as support for the crystals of the reactive phase which is sure to form. Their mineral nature, goethite, lepidocrocite, ferrihydrite etc., is unimportant since bacterial reduction replaces the initial ferric oxyhydroxy by dissolution-precipitation.

The invention can be used in a lysimeter installed at an appropriate site.

In fact, the high levels of nitrates in Brittany are responsible for the problems relating to the proliferation of green algae. Thus, the process of catalytic reduction of nitrates according to the invention can be used in places recently contaminated with green algae, such as Trestel, or places that are completely polluted, such as the shore of the bay of Saint Michel at Grève.

### DESCRIPTION OF THE DRAWINGS

FIG. 1: Schematic diagram of the principle of operation of the catalyst according to the invention, the ferrous-ferric oxyhydroxy salt, for the implementation of an oxidation-reduction process permitting the reduction of a substance S.

The dissimilatory iron-reducing bacteria oxidize the organic matter (CH₂O) in the course of their respiration to CO₃²⁻ (1). The final electron acceptor of the bacterial respiratory chain is represented by Fe^{III}, which is thus reduced to Fe^{II} actually within the catalyst (the ferrous-ferric oxyhydroxy salt).

This oxidation reaction of the organic matter is coupled to a reduction of a substance S to a substance S_{reduced} by oxidation of Fe^{II} to Fe^{III} (2).

Fe^{III} resulting from the reduction of the substance S thus constantly regenerates the Fe^{II} via the bacterial respiration and the catalyst is perpetuated, during a lithotrophic catalytic cycle (3).

The organobacterial catalytic oxidation is therefore coupled to the catalytic reduction of the substance S.

The grey elements relate to the catalyst, the ferrous-ferric oxyhydroxy salt, within which Fe^{III} represents the oxidizing catalytic site (the anode) and Fe^{II} represents the reducing catalytic site (the cathode).

FIG. 2: Micrograph obtained with a scanning electron microscope (SEM) showing iron-reducing bacteria (Shewanella putrefaciens) during respiration in contact with ferrous-ferric oxyhydroxycarbonate. The bacteria attach themselves to the crystal of hexagonal-base prismatic form with filaments facilitating the transfer of electrons. At some stage a biofilm is created.

FIG. 3: Principle of catalytic reduction of nitrates according to the invention.

The dissimilatory iron-reducing bacteria (DIRB) oxidize the organic matter (CH₂O) to CO₃²⁻ in the course of their respiration (1). The final electron acceptor of the bacterial respiratory chain is represented by Fe^{III}, which is thus reduced to Fe^{II} actually within the catalyst (the ferrous-ferric oxyhydroxycarbonate).

The bacterial respiration is coupled to a reduction of the nitrates (NO₃⁻) by oxidation of Fe^{II} to Fe^{III} (2). The nitrates are reduced to dinitrogen (N₂), permitting for example the denitrification of a medium.

The Fe^{III} resulting from reduction of the nitrates thus constantly regenerates the Fe^{II} via the bacterial respiration and the catalyst is perpetuated, during a lithotrophic catalytic cycle (3).

The organobacterial catalytic oxidation is then coupled to a catalytic reduction of the nitrates.

The grey elements relate to the catalyst, here the ferrous-ferric oxyhydroxycarbonate, within which Fe^{III} represents the oxidizing catalytic site (the anode) and Fe^{II} represents the reducing catalytic site (the cathode).

FIG. 4: Column reactor used in the laboratory for evaluating the reduction of the substance S by the catalyst according to the process of the invention.

The reactor used comprises a column (4) of about 60 cm which is filled with a model medium, under conditions of anoxia. This medium comprises in particular the catalyst according to the invention, iron-reducing bacteria and organic matter.

The liquid solution to be treated, containing the substance S, is fed into the system at the inlet (1) flowing to a tank (2) which is fitted with measuring electrodes (pH and potential) and a gas inlet (N₂ and O₂). The gas inlet is used for controlling the conditions of anoxia.

The liquid solution to be treated is driven by a peristaltic pump (3) through the column in ascending mode. At the column outlet, the treated medium is delivered to an analysis chamber (7) which evaluates the effectiveness of the process, then to the outlet (1), where the medium is either recovered, or recycled to the system.

MIMOS (5) is a miniaturized Mössbauer spectrometer constructed at the University of Mayence (Dr. G. Klingerhöffer). It is a clone of the miniaturized Mössbauer spectrometer probe sent to Mars to analyse the iron oxides there (NASA and ESA programmes). MIMOS permits semi-continuous in situ analysis of the ratio x=Fe^{III}/Feₜₒₜₐₗ inside the catalyst.

Oz indicates the vertical axis.

At the outlet (6) of the system, samples of solution can be taken and various measurements can be carried out.

The column can also be used in descending flow.

FIG. 5: Photographs of a support of the sand type (polycrystalline silica) coated with the catalyst precursor, ferric oxyhydroxycarbonate, of formula [Fe^{II}₆O₁₂H₈]²⁺CO₃²⁻ according to the invention.

The terms “dry”, “in solution”, and “during synthesis” correspond to the possible types of deposition, respectively dry preparation, preparation in solution and preparation during synthesis.

The catalyst deposit corresponds to the whitish areas discernible on the surface of the grain of sand at high magnification. The quality of this coating is a key element of the process.

FIG. 6: Mössbauer spectrum measured in situ by means of MIMOS on the surface of sand coated with ferric oxyhydroxycarbonate, clearly identifying the presence of the latter exclusively, by comparing the intensity of the peaks according to the type of preparation of the coating.

FIG. 7: Mössbauer spectra measured in situ under ambient conditions by reflection with MIMOS:

FIG. 7(a): GR(CO₃²⁻) initial at x=0.33,

FIG. 7(b): GR(CO₃²⁻)* at x=0.38 after oxidation by the nitrates, 1 day,

FIG. 7(c): GR(CO₃²⁻)* at x=0.58 after oxidation by the nitrates, 11 days,

FIG. 7(d): Magnetite+GR(CO₃²⁻)* at x=1 after oxidation by the nitrates, 1 month.

FIG. 8: Electrode potential as a function of time, of a solution containing hydroxycarbonate into which a nitrogen-oxygen stream is bubbled, while stirring at 375 rpm.

FIG. 8(a): Increase in the proportion of oxygen (air) in an N₂—O₂ stream. The proportion of oxygen is 2.7%, 6.7%, 13.3% and 20% respectively, for the four curves from right to left. The circled letters B and C correspond to the plateaux reached.

(G=goethite and M=magnetite)

FIG. 8(b): Oxidation in the Air with Stirring at 1500 Rpm and a pH of 7 (Bottom Curve) or 9 (top curve) (GR*=oxyhydroxycarbonate).

FIG. 8(c): Representation on the same scale of the kinetics obtained in FIG. 8(b) (curve on the left) and in FIG. 8(a) (middle curve, proportion of O₂=20% and curve on the right, proportion of O₂=6.7%).

FIG. 9: X-ray diffraction and Mössbauer spectrometry of the products obtained in Example 4:

FIGS. 9a and 9c: X-ray diffraction and Mössbauer spectrometry, respectively, of the product in FIG. 8a (20% O₂),

FIGS. 9b and 9d: X-ray diffraction and Mössbauer spectrometry, respectively, of the product in FIG. 8b.

### EXAMPLES

The experiments relating to the present invention are divided into five operational phases combining applied research in the laboratory, experimentation in the testing facilities and field demonstrator.

The first example relates to a preferred embodiment of the preparation of the catalyst.

The second example relates to the development of a novel mild chemical process using a synthetic catalyst, where denitrification takes place in a closed installation, with the possibility of provision in several versions and formats as required.

### Example 1

### Preparation of the Catalyst

### Materials and Methods

a) Catalyst Precursor without Substrate The ferrous-ferric hydroxycarbonate [Fe^{II}₄Fe^{III}₂(OH)₁₂]²⁺CO₃²⁻ is prepared by chemical synthesis, either by oxidation of a precipitate of Fe(OH)₂ in the presence of carbonate ions as described by Génin et al. (2006, Geoscience), or by co-precipitation of Fe^{II} and Fe^{III} ions in the presence of anions as described by Ruby et al. (2006, Geoscience). This ferrous-ferric hydroxycarbonate is then completely deprotonated with a vigorous oxidizing agent such as H₂O₂ in excess or in air after drying, as described in Genin et al. (2006, Geoscience) in order to form the ferric oxyhydroxycarbonate of formula [Fe^{III}₆O₁₂H₈]²⁺ CO₃²⁻ which will serve as precursor for the ferrous-ferric oxyhydroxycarbonate catalyst of general formula [Fe^{II}₆₍₁₋ₓ₎Fe^{III}₆ₓO₁₂H₂₍₇₋₃ₓ₎]²⁺CO₃²⁻ in the range where x is between 0.33 and 0.66.

The transition from catalyst precursor to catalyst takes place later during start-up by bacterial reduction in situ without modification of structure or morphology.

The product obtained is characterized by X-ray diffraction, Mössbauer spectrometry, vibrational spectrometry (Raman or infrared), transmission electron microscopy.

### b) Catalyst-Coated Support

Coating with the catalyst precursor, here ferric oxyhydrocarbonate [Fe^{III}₆O₁₂H₈]²⁺CO₃²⁻, is obtained by “dry” or “in solution” deposition of the precursor on a support, or by adding the support at the same time that the precursor is synthesized.

The protocol for “dry” preparation is as follows:
coprecipitation in solution of Fe^{II} and Fe^{III} ions in the presence of carbonate anions, in order to obtain the ferrous-ferric hydroxycarbonate of formula

[Fe^{II}₄Fe^{III}₂(OH)₁₂]²⁺CO₃²⁻,
complete and rapid oxidation, by H₂O₂ in excess in the solution or in the air after drying of said dry ferrous-ferric hydroxycarbonate, in order to obtain the precursor: the ferric oxyhydroxycarbonate of formula

[Fe^{III}₆O₁₂H₈]²⁺CO₃²⁻,
filtration then complete drying of said ferric oxyhydroxycarbonate, in order to obtain a dry ferric oxyhydroxycarbonate, and
mechanical mixing of the dry ferric oxyhydroxycarbonate with said substrate, in order to obtain said substrate coated with ferric oxyhydroxycarbonate, and
washing of the support and its deposit with distilled water.

The protocol for the “in solution” preparation is as follows:
coprecipitation in solution of Fe^{II} and Fe^{III} ions in the presence of carbonate anions, in order to obtain the ferrous-ferric hydroxycarbonate of formula

[Fe^{II}₄Fe^{III}₂(OH)₁₂]²⁺CO₃²⁻,
complete and rapid oxidation of said ferrous-ferric hydroxycarbonate by H₂O₂ in solution, in order to obtain the ferric oxyhydroxycarbonate of formula

[Fe^{III}₆O₁₂H₈]²⁺CO₃²⁻,
addition of said substrate to said solution, in order to obtain a substrate coated with the ferric oxyhydroxycarbonate in solution, and

filtration and drying of said substrate coated with the ferric oxyhydroxycarbonate in solution, in order to obtain said substrate coated with the precursor, dry ferric oxyhydroxycarbonate.

In “dry” preparation and “in solution” preparation, the conditions of the test described relate to about a hundred grams of sand with a few grams of dry ferric oxyhydroxycarbonate. Mixing is carried out without particular precautions, at room temperature, since the precursor is totally ferric and therefore there is no risk of further oxidation. The excess of precursor is recovered and it is very important to have a volume ratio of support to precursor of about 1000. Once it is coated with the precursor, the support is washed with distilled water. However, the precursor remains attached to the surface of the support, as can be seen in the micrograph in FIG. 5 as a very fine layer (but difficult to evaluate), which promises good effectiveness for the future catalyst, which must have a large developed surface.

The protocol for preparation during synthesis of the catalyst is as follows:
introducing Fe^{II} and Fe^{III} ions, carbonate anions, H₂O₂ and said substrate in solution, and
coprecipitation of said Fe^{II} and Fe^{III} ions in the presence of carbonate anions and immediate simultaneous oxidation by H₂O₂, in order to obtain said substrate coated with the ferric oxyhydroxycarbonate of formula [Fe^{III}₆O₁₂H₈]²⁺CO₃²⁻.

### Results

The first protocol with the ferric oxyhydroxycarbonate dried and deposited dry on the grains of sand gives the best result, as is clear from the intensity of the peaks obtained by Mössbauer spectrometry (FIG. 6). A larger amount of iron is deposited, the layer of precursor is thicker and its distribution is more uniform.

It was also verified that its quality is maintained in a reactor at the end of the reaction.

### Example 2

### Development of a Mild Chemical Process for Denitrification in a Closed Installation

Phase 1: Experimentation in the Laboratory on a Reduced Amount of Reactive Material (about 1 kg)

### Materials and Methods

The support (sand or clays) coated with the precursor ferric oxyhydroxycarbonate (x=1) is prepared as described in Example 1.

a) Obtaining the catalyst

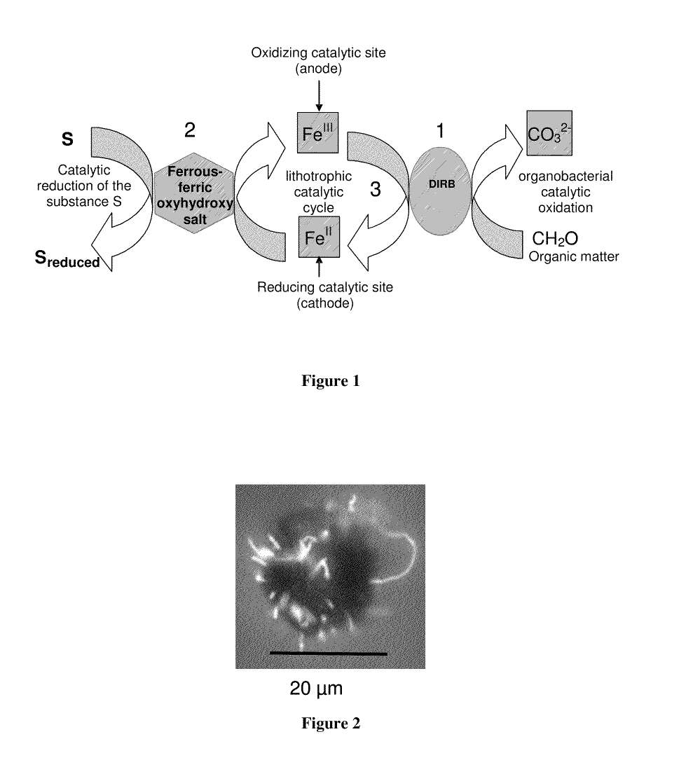

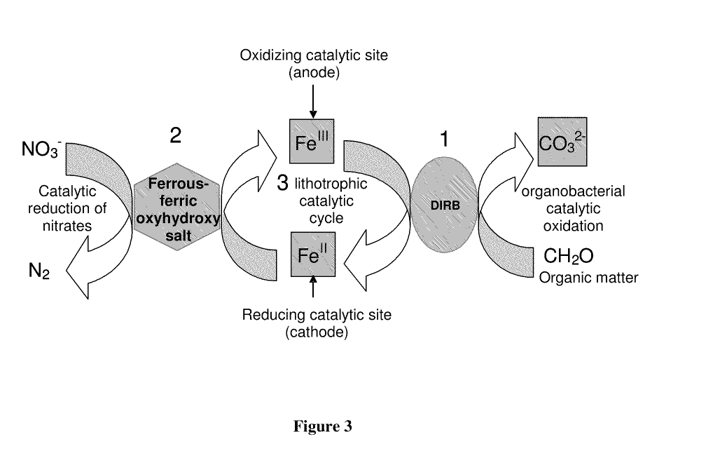

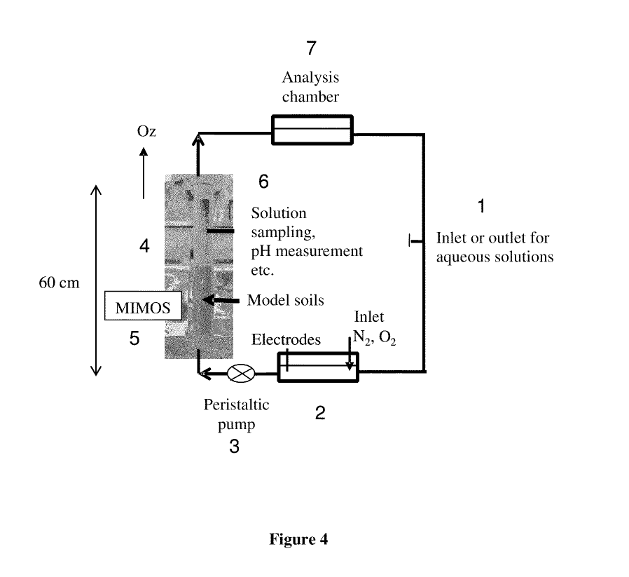

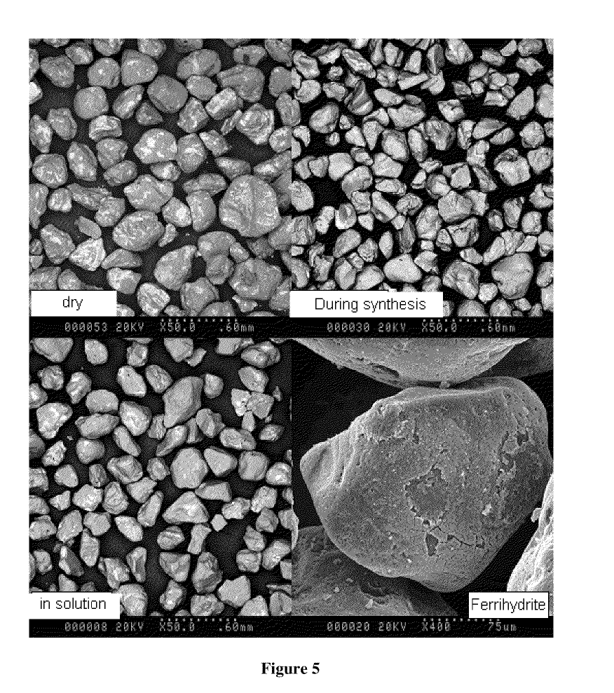

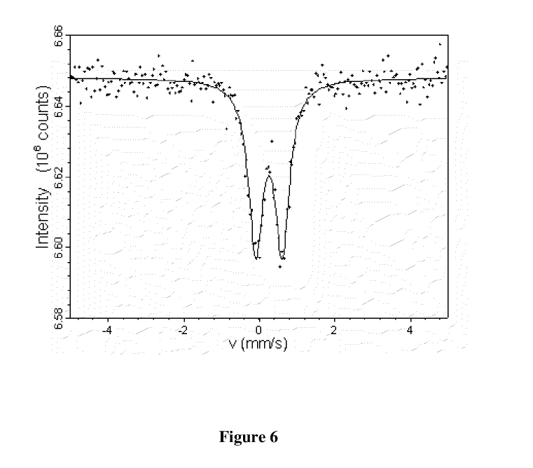

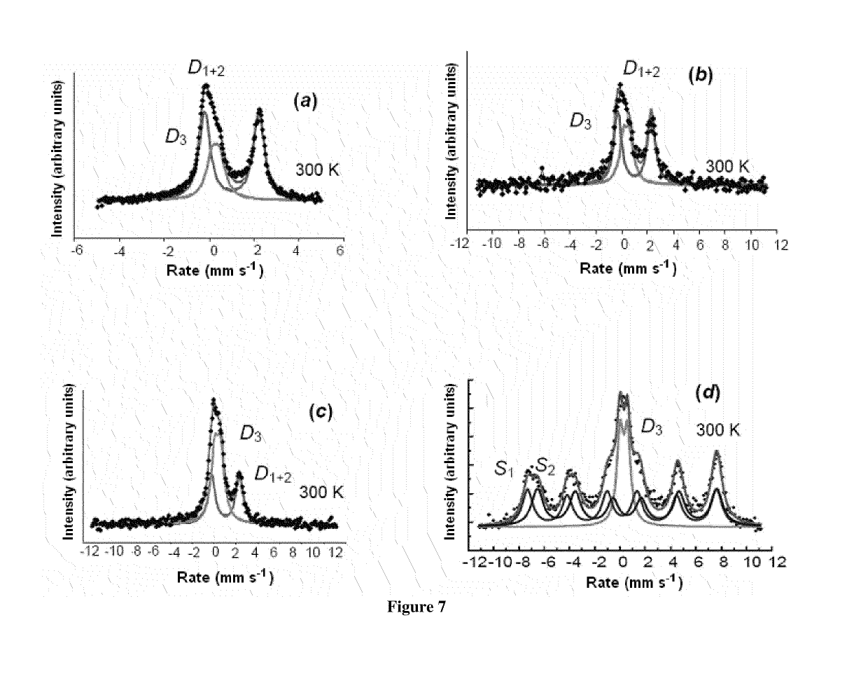

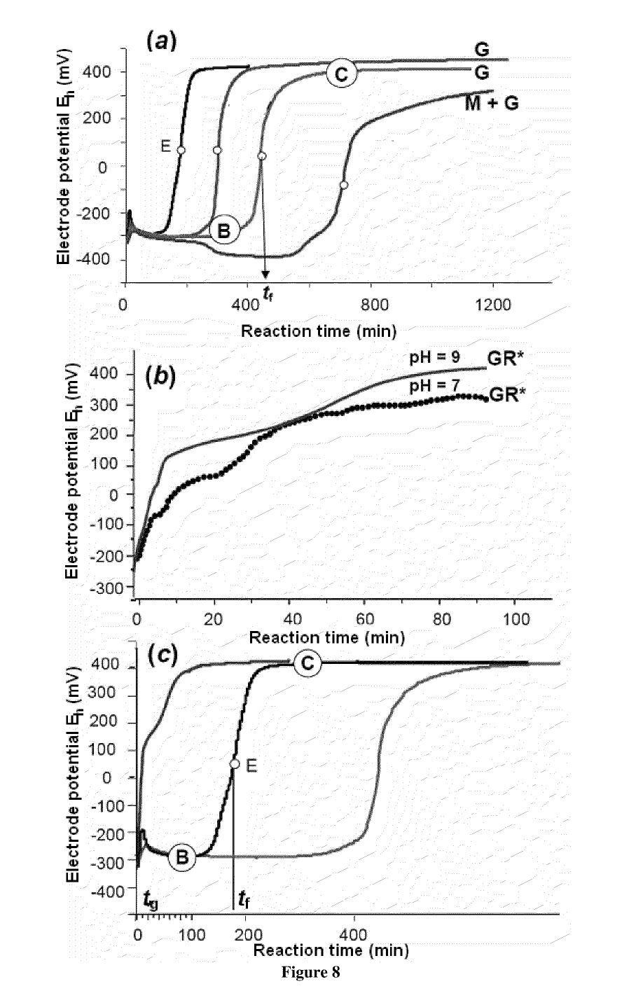

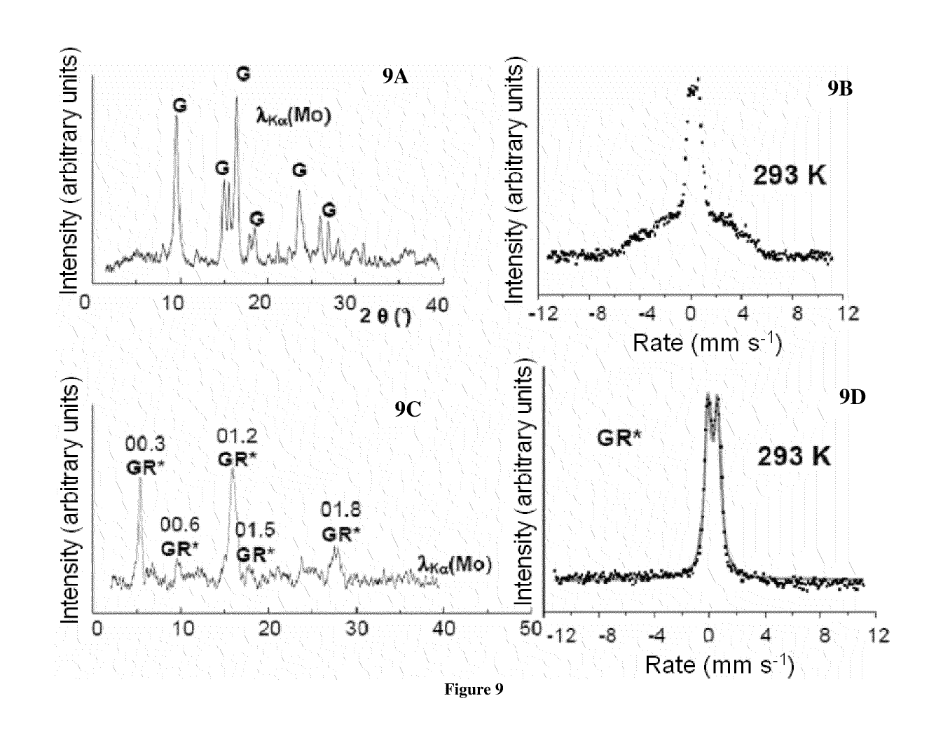

After manufacture of the precursor on its support, the latter is dissolved again with the iron-reducing bacteria and the organic matter, so that bacterial reduction of Fe^{III} to Fe^{II} takes place actually within the precursor (FIGS. 1 and 2). In contrast to the case when the ferric species would be those of another precursor, any ferric oxyhydroxide FeOOH, in this case there is no dissolution of the precursor then reprecipitation of the catalyst elsewhere. The catalyst that forms remains physically where the deposit of precursor on the support was attached. This is essential, since the surface layer morphology on the sand grains is preserved and the optimum arrangement sought for catalysis is effectively obtained at the end of manufacture of the precursor. Now, this arrangement is not necessarily that observed in natural soils. By using the fully oxidized form with the same structure as the catalyst as precursor, the process in the reactor is a priori more efficient than what occurs under natural conditions.

The formation of the catalyst in situ starting from the precursor is monitored in situ with the MIMOS spectrometer (Miniaturized Mössbauer Spectrometer). There is no intervention, since the γ rays used for measuring the spectra pass through the wall of the reactor. Characterization is therefore semi-continuous (a spectrum may require one day of recording, whereas the process of bacterial reduction takes of the order of a week in a beaker).

b) Catalytic reduction of nitrates

The kinetics of reduction of the nitrates in solution by the catalyst was investigated using the device shown in FIG. 4.

The waters used in the experiment are spring waters of various kinds, doped with nitrates to simulate well waters. The nitrate content is typically fixed at 100 mg/l. Several flow rates are tested in order to ascertain the operating limits

Waters used after settling and coagulation/flocculation treatment (water laden with DCO, MO, nitrates) are also tested to simulate the case of municipal wastewaters after secondary treatment or waters from septic tanks.

All these tests included monitoring of the various parameters for evaluating the effectiveness of the treatment, in addition to the conventional monitoring of pH, temperature, and oxidation-reduction potential; in particular monitoring, continuously as far as possible, of the concentrations of the various species formed is systematically investigated: analyses of the nitrogen-containing and carbon-containing species, and species of iron, where Mössbauer reflection spectrometry, MIMOS, provides the Fe^{III}/Feₜₒₜₐₗ ratio observed in situ in the catalyst.

### Results

The nitrogen concentration is analysed in its nitrate, gaseous nitrogen, nitrite, and ammonium forms. The results obtained allow the conclusion that the presence of the nitrite and ammonium forms is negligible.

The results obtained show that the catalytic product permits reduction of the nitrates present in a medium to be treated.

### Phase 2: Experimentation in the Testing Facility on a Significant Amount of Reactive Product

### Materials and Methods

The study relates to the use of an amount of the order of 100 kg of ferrous-ferric oxyhydroxy salt, in a column at the pilot-plant scale of the NanCIE technology platform (Centre International de l'Eau de Nancy) at Laneuveville-devant-Nancy (Laneuveville near Nancy).

The protocol for monitoring and characterization of the species is identical to that for the first phase. In particular, the behavior and the variation over time of the reactive coating are analysed to evaluate the durability of the process employed.

The Laneuveville site permits treatment of three broad categories of water:
drinking water,
well water,
river water particularly saline, and
conventional municipal wastewater.

These waters are characterized for nitrate content and doped according to the required concentrations.

Phase 3: Experimentation on Site in Brittany for Reduction of the Nitrate Level: Production of Drinking Water, Treatment of Wastewaters (Perros-Guirec or Trégastel)

The three pilot studies in Brittany, permitting a decrease in nitrates, are as follows:
production of drinking water: the experiment relates to a production rate of about 10 m³/day, corresponding to 50 equivalent inhabitants,
semi-collective sanitation of the septic tank type, with supplementary treatment of the water before infiltration in the soils (scattered rural settlement),
treatment of municipal wastewaters adapted to small treatment works (activated sludges or lagooning) deficient in the treatment of nitrogen, the flow treated being equivalent to 10 m³/day.

Dimensioning for treatment equivalent to about fifty houses is the aim. Reactors of this type were developed in the testing facility of the technology platform of Laneuveville-devant-Nancy by NanCIE.

It was verified that the reduction reaction of the nitrates is fully mastered and leads only to the formation of gaseous nitrogen, and there is no formation of nitrite or ammonium.

### Example 3

### Deprotonation of Ferrous Oxyhydroxycarbonate (GR) During Reduction of the Nitrates and the Role of Copper and of Phosphate

A mixture of FeSO₄-7H₂O and of Fe₂SO₄-5H₂O salts is dissolved in 100 mL of demineralized water ([Fe]=0.4 M) with continuous bubbling with N₂.

GR(CO₃²⁻) at x=0.33 is precipitated by progressively adding a solution of Na₂CO₃ to the initial mixture until the pH reaches a value of 9.5. Then a small quantity of Na₂HPO₄.12H₂O and CuSO₄.5H₂O salts is dissolved in the suspension ([PO₄]=4×10⁻³ M and [Cu^{II}]=4×10⁻² M). The phosphate anions are used for stabilizing the GR structure. The Cu^{II} cations are added in order to accelerate the kinetics of oxidation as proposed by Ottley et al., who investigated the reduction of the nitrate by ferrous hydroxide.

At this stage, the stoichiometric GR(CO₃²⁻) has the formula [Fe^{II}₄Fe^{III}₂(OH)₁₂]²⁺.[CO₃².3H₂O]². Oxidation of GR(CO₃²⁻) begins when a solution of NaNO₃ ([NO₃⁻]=0.8 M) is added to the suspension. The reaction takes less than one month.

Samples of the precipitates are taken periodically by filtration under an N₂ atmosphere. They are introduced into a support to permit their characterization using the reemission of γ radiation of 14.4 keV with a miniaturized Mössbauer spectrometer (MIMOS), at room temperature, with back-reflection geometry.

### Results:

The results are presented in FIG. 7 and Table 1:

TABLE 1
Mössbauer hyperfine parameters measured at ambient temperature on samples
of GR(CO₃²⁻) that are oxidized in situ by nitrates: FIG. (7a) initial GR(CO₃²⁻), FIG.
(7b) 1 day in the presence of NO₃⁻, FIG. (7c) 11 days in the presence of NO₃⁻, FIG.
(7d) 1 month in the presence of NO₃⁻.

| | GR(CO₃²⁻) | GR(CO₃²⁻)* | GR(CO₃²⁻)* | GR(CO₃²⁻)* |
|---|---|---|---|---|
| x | 0.33 | 0.38 | 0.58 | 1 |
| FIG. | 7a | 7b | 7c | 7d |
| T | 300 K | 300 K | 300 K | 300 K |

| | δ | Δ | RA | δ | Δ | RA | δ | Δ | RA | δ | Δ | H | RA |
|---|---|---|---|---|---|---|---|---|---|---|---|---|---|
| | (mm s⁻¹) | | (%) | (mm s⁻¹) | | (%) | (mm s⁻¹) | | (%) | (mm s⁻¹) | | (kOe) | (%) |
| D₁₊₂ | 1.12 | 2.4 | 67 | 0.98 | 2.6 | 62 | 1.03 | 2.53 | 42 | | | | |
| D₃ | 0.41 | 0.35 | 33 | 0.37 | 0.49 | 38 | 0.31 | 0.55 | 58 | 0.40 | 0.57 | | 20 |
| S₁ | | | | | | | | | | 0.27 | | 463 | 39 |
| S₂ | | | | | | | | | | 0.67 | | 440 | 41 |

δ: isomer shift in mm s⁻¹ (Reference: metallic α Fe at room temperature);
Δ: quadrupole division in mm s⁻¹;
H: hyperfine field in kOe;
RA: relative proportion in %.
The half-width at mid-height is about 0.7 mm s⁻¹.

Two quadrupole doublets only originate from oxyhydroxycarbonate GR(CO₃²⁻)* with D₁₊₂ (Fe^{II}) and D₃ (Fe^{III}). The intensity of D₃ directly gives x=0.33, 0.38 and 0.58. (d) mixture of magnetite and ferric GR*.

Oxidation takes place in situ until beyond x=0.67 GR* partially decomposes to magnetite. A portion of the oxyhydroxycarbonate nevertheless reaches x=1.

Copper notably accelerates reduction of the nitrates, and phosphate stabilizes green rust vis-à-vis magnetite.

Without copper, the reaction is particularly slow (˜two orders of magnitude). Cu therefore performs the role of catalyst vis-à-vis iron.

### Example 4

### Study of the Deprotonation of Ferrous Oxyhydroxycarbonate (GR)

As the conditions for oxidation in situ by deprotonation and by dissolution-reprecipitation lead to the same rusts, ferric oxyhydroxides free from carbonates such as ferrihydrite, lepidocrocite or goethite were investigated.

In particular, the oxygen flow was increased progressively and this made it possible to change over from one operating mode to another.

This is illustrated by FIG. 8, which shows the electrode potential as a function of time in the beaker, with magnetic bar stirring of a solution containing hydroxycarbonate, into which a nitrogen-oxygen mixture is bubbled:

In FIG. 8a, the oxygen level is increased (from right to left) from 2.7-6.7-13.3 to 20% (air) at an N₂—O₂ flow of 2.3×10⁻³ L s⁻¹ with constant stirring with the magnetic bar at 375 rpm. There are two plateaux B and C, which correspond to the progressive oxidation of the green rust to ferrihydrite and then goethite. Among other things, it is noted that on increasing the proportion of oxygen, the oxidation characterized by the equivalent point E becomes quicker and quicker, changing from about 700 min to 200 min. The first curve, the slowest at 2.7% O₂, gives magnetite and goethite as product, showing that in that case Fe²⁺ remains as it is and is incorporated in the solid, thus forming magnetite.

At the start of the first plateau B, a hook-shape appears, becoming more and more pronounced (see below) when the proportion of oxygen increases.

In FIG. 8b: oxidation in air but with stirring 4 times as fast: 1500 rpm instead of 375. Two experiments are carried out: pH9 and pH7. The electrode potential increases continuously, which is characteristic of oxidation in situ with the formation of the oxyhydroxycarbonate, with x increasing progressively from 0.33 to 1. The curve is entirely similar to that obtained when oxidizing with H₂O₂. The half-reaction time does not exceed 10 min, compared with the previous 200 min Consequently, deprotonation in situ without salting-out of the carbonates from the solid is much quicker than dissolution-reprecipitation. The role of the pH is rather insignificant.

In FIG. 8c: this shows the kinetics of the two modes of oxidation on the same time scale. This superposition of the curves provides an explanation for the hook-shape mentioned above. Even when oxidation is not very vigorous, at the start it takes place in situ until dissolution-reprecipitation becomes dominant. It is a problem of kinetics.

FIG. 9 clearly shows that the final oxidation products are goethite G for the slow process and oxyhydroxycarbonate GR* for vigorous oxidation. X-ray diffraction and Mössbauer spectrometry are in full agreement. Moreover, it is observed that goethite is superparamagnetic, i.e. it has very small crystals.

This study shows that the kinetics of oxidation in situ is much quicker than the more traditional oxidation and that it can be carried out simply with air.

1-39. (canceled)

40. Method for the implementation of an oxidation-reduction process by means of at least one lamellar double hydroxide (LDH) as catalyst or as precursor of said catalyst, with the same crystalline structure as that of said catalyst, said LDH containing a divalent cation M²⁺ partially or completely substituted with Fe^{II}, and a trivalent cation T³⁺ optionally substituted with Fe^{III}, of the following general formula:
[M²⁺_{(z)}Fe^{II}_{(1-y-z)}T³⁺ₜFe^{III}_{(y-t)}O₂H₂]ⁿ⁺[(y/n)Aⁿ⁻,mH₂O]ⁿ⁻,
in which:
¼<y<⅓, z<1−y and t<y, Aⁿ⁻ is an anion with charge n, n having the values 1, 2 or 3, m is an integer varying from 1 to 10,
and the ratio x=(y−t)/(1−z−t) can vary from 0 to 1,
said LDH being used in association with iron-reducing bacteria that are able to reduce Fe^{III} to Fe^{II} and in the presence of organic matter, and can be deprotonated to give the following formula:
[M²⁺_{(z)}Fe^{II}_{(1-y-z-w)}T³⁺ₜFe^{III}_{(y-t+w)}O₂H_{2-w}]ⁿ⁺[(y/n)Aⁿ⁻,mH₂O]ⁿ⁻,
in which:
A, y, z, m and n are as above,
and the ratio x=(y−t+w)/(1−z−t) can vary from 0 to 1, in order to reduce a substance S to a substance S_{reduced}, the redox potential of the pair S_{reduced}/S being greater than that of the pair Fe^{II}/Fe^{III} at the crystallographic sites of the Fe^{II}, x varying essentially in the range from 0.33 to 0.66 after the start-up of the oxidation-reduction process, and without a substantial change in the crystalline structure of the aforesaid LDH.

41. The method according to claim 40, in which the proportion of Fe^{II} substituting the divalent element is comprised from 1% (w/w) to 100% (w/w) relative to the total amount of divalent element.

42. The method according to claim 40, in which the proportion of Fe^{III} in the trivalent element is comprised from 0% (w/w) to 100% (w/w) relative to the total amount of trivalent element.

43. The method according to claim 40, in which M²⁺ is selected from Mg²⁺, Ni²⁺, Ca²⁺, Mn²⁺, and T³⁺ is selected from Al³⁺ and Cr³⁺.

44. The method according to claim 40, wherein the LDH is in the form of a ferrous-ferric oxyhydroxy salt as catalyst or as precursor of said catalyst, with the same crystalline structure as that of said catalyst, for the implementation of an oxidation-reduction process, said ferrous-ferric oxyhydroxy salt having the formula
[Fe^{II}₃ₙ₍₁₋ₓ₎Fe^{III}₃ₙₓO₆ₙHₙ₍₇₋₃ₓ₎]ⁿ⁺[Aⁿ⁻,mH₂O]ⁿ⁻
in which Aⁿ⁻ is an anion with charge n, n having the values 1, 2 or 3, m is an integer varying from 1 to 10, and x is in the range from 0 to 1,
said ferrous-ferric oxyhydroxy salt being used in association with iron-reducing bacteria that are able to reduce Fe^{III} to Fe^{II} and in the presence of organic matter, in order to reduce a substance S to a substance S_{reduced}, the redox potential of the pair S_{reduced}/S being greater than that of the pair Fe^{II}/Fe^{III} at the crystallographic sites of the Fe^{II}, x varying essentially in the range from 0.33 to 0.66 after the start-up of the oxidation-reduction process, without a substantial change in the crystalline structure of the aforesaid ferrous-ferric oxyhydroxy salt.

45. The method according to claim 40, for the implementation of a process in which the substance S is reduced to a substance S_{reduced} by oxidation of Fe^{II} to Fe^{III} and in which the organic matter is oxidized at the end of the reduction of Fe^{III} to Fe^{II} by the iron-reducing bacteria.

46. The method according to claim 40, in which the substance S is selected from inorganic pollutants including nitrate, selenate, chromate, arsenate or from organic pollutants.

47. The method according to claim 40, in which the bacteria are selected from the genera Shewanella putrefaciens, Geobacter sp.

48. The method according to claim 40, wherein the LDH is in association with a metal selected from Cu(II), Ag(I), Cd(II), Ni(II), Hg(II), Pb(II) and Mn(II), preferably Cu(II), in a proportion from 2 to 20% (w/w) relative to the total Fe.

49. The method according to claim 40, wherein the LDH is in association with phosphate ions in a proportion of at least 1%.

50. Process permitting the reduction of a substance S to a substance S_{reduced} comprising:
introducing a LDH, as catalyst or precursor of said catalyst with the same crystalline structure as that of said catalyst,
said LDH containing a divalent cation M²⁺ partially or completely substituted with Fe^{II}, and a trivalent cation T³⁺ optionally substituted with Fe^{III}, of the following general formula:
[M²⁺_{(z)}Fe^{II}_{(1-y-z)}T³⁺ₜFe^{III}_{(y-t)}O₂H₂]ⁿ⁺[(y/n)Aⁿ⁻,mH₂O]ⁿ⁻
in which:
¼<y<⅓, z<1−y and t<y, Aⁿ⁻ is an anion with charge n, n having the values 1, 2 or 3, m is an integer varying from 1 to 10,
and the ratio x=(y−t)/(1−z−t) varies from 0 to 1,
said LDH being used in association with iron-reducing bacteria able to reduce Fe^{III} to Fe^{II} and in the presence of organic matter,
if x is greater than 0.66 at the initial moment, a start-up phase of the oxidation-reduction process corresponding to the reduction of Fe^{III} to Fe^{II} within said LDH by said iron-reducing bacteria, leading to a change in x to a value less than or equal to 0.66, in order to obtain said LDH in the form of a catalyst, without a substantial change in the crystalline structure of said LDH,
a phase of catalytic reduction of the substance S, added to the whole comprising the LDH, the bacteria and the organic matter, to a substance S_{reduced} by oxidation of the Fe^{II} to Fe^{III} within said LDH coupled to a stage of catalytic oxidation of the organic matter by reduction of the Fe^{III} to Fe^{II}, the redox potential of the pair S_{reduced}/S being greater than that of the pair Fe^{II}/Fe^{III} at the crystallographic sites of the Fe^{II}.

51. The process permitting the reduction of a substance S to a substance S_{reduced} according to claim 50, in which said LDH is in the form of a ferrous-ferric oxyhydroxy salt and comprising:
introducing said ferrous-ferric oxyhydroxy salt, as catalyst or precursor of said catalyst with the same crystalline structure as that of said catalyst, having the formula
[Fe^{II}₃ₙ₍₁₋ₓ₎Fe^{III}₃ₙₓO₆ₙHₙ₍₇₋₃ₓ₎]ⁿ⁺[Aⁿ⁻,mH₂O]ⁿ⁻
in which Aⁿ⁻ is an anion with charge n, n having the values 1, 2 or 3, m is an integer varying from 1 to 10, and x is in the range from 0 to 1 at the initial moment,
with iron-reducing bacteria that are able to reduce the Fe^{III} to Fe^{II} and organic matter,
if x is greater than 0.66 at the initial moment, a start-up phase of the oxidation-reduction process corresponding to the reduction of Fe^{III} to Fe^{II} within said ferrous-ferric oxyhydroxy salt by said iron-reducing bacteria, leading to a change in x to a value less than or equal to 0.66, in order to obtain said ferrous-ferric oxyhydroxy salt in the form of a catalyst, without a substantial change in the crystalline structure of said ferrous-ferric oxyhydroxy salt,
a phase of catalytic reduction of the substance S, added to the whole comprising the ferrous-ferric oxyhydroxy salt, the bacteria and the organic matter, to a substance S_{reduced} by oxidation of the Fe^{II} to Fe^{III} within said ferrous-ferric oxyhydroxy salt coupled to a stage of catalytic oxidation of the organic matter by reduction of the Fe^{III} to Fe^{II},
the redox potential of the pair S_{reduced}/S being greater than that of the pair Fe^{II}/Fe^{III} at the crystallographic sites of the Fe^{II}.

52. The process permitting the reduction of a substance S to a substance S_{reduced} according to claim 50, in which said LDH is used in association with a metal selected from Cu(II), Ag(I), Cd(II), Ni(II), Hg(II), Pb(II) and Mn(II), preferably Cu(II), in a proportion from 2 to 20% (w/w) relative to the total Fe.

53. The process permitting the reduction of a substance S to a substance S_{reduced} according to claim 50, in which said LDH is used in association with phosphate ions in a proportion of at least 1%.

54. Process permitting the reduction of a substance S to a substance S_{reduced} comprising:
introducing a LDH, as catalyst precursor, said LDH containing a divalent cation M²⁺ partially or completely substituted with Fe^{II}, and a trivalent cation T³⁺ optionally substituted with Fe^{III}, of the following general formula:
[M²⁺_{(z)}Fe^{II}_{(1-y-z)}T³⁺ₜFe^{III}_{(y-t)}O₂H₂]ⁿ⁺[(y/n)Aⁿ⁻,mH₂O]ⁿ⁻
in which:
¼<y<⅓, z<1−y and t<y, Aⁿ⁻ is an anion with charge n, n having the values 1, 2 or 3, m is an integer varying from 1 to 10,
and the ratio x=(y−t)/(1−z−t) can vary from 0 to 1, said LDH being used in association with iron-reducing bacteria able to reduce Fe^{III} to Fe^{II} and in the presence of organic matter,
if x is greater than 0.66 at the initial moment, a start-up phase of the oxidation-reduction process corresponding to the reduction of Fe^{III} to Fe^{II} within said LDH by said iron-reducing bacteria, leading to a change in x to a value less than or equal to 0.66, in order to obtain said LDH in the form of a catalyst, without a substantial change in the crystalline structure of said LDH,
a phase of catalytic reduction of the substance S, added to the whole comprising the LDH, the bacteria and the organic matter, to a substance S_{reduced} by oxidation of the Fe^{II} to Fe^{III} within said LDH coupled to a stage of catalytic oxidation of the organic matter by reduction of the Fe^{III} to Fe^{II}, the redox potential of the pair S_{reduced}/S being greater than that of the pair Fe^{II}/Fe^{III} at the crystallographic sites of the Fe^{II}.

55. The process permitting the reduction of a substance S to a substance S_{reduced} according to claim 54 in which said LDH is in the form of a ferrous-ferric oxyhydroxy salt and comprising:
introducing said ferrous-ferric oxyhydroxy salt as catalyst precursor having the formula
[Fe^{II}₃ₙ₍₁₋ₓ₎Fe^{III}₃ₙₓO₆ₙHₙ₍₇₋₃ₓ₎]ⁿ⁺[Aⁿ⁻,mH₂O]ⁿ⁻
in which Aⁿ⁻ is an anion with charge n, n having the values 1, 2 or 3, m is an integer varying from 1 to 10, advantageously 3, and x is greater than 0.66 at the initial moment,
with iron-reducing bacteria that are able to reduce the Fe^{III} to Fe^{II} and organic matter,
a phase of process start-up corresponding to the reduction of Fe^{III} to Fe^{II} within said ferrous-ferric oxyhydroxy salt by said iron-reducing bacteria, leading to a change in x to a value less than or equal to 0.66, in order to obtain said ferrous-ferric oxyhydroxy salt in the form of a catalyst, without a substantial change in the crystalline structure of said ferrous-ferric oxyhydroxy salt,
a phase of catalytic reduction of the substance S, added to the whole comprising the ferrous-ferric oxyhydroxy salt, the bacteria and the organic matter, to a substance S_{reduced} by oxidation of the Fe^{II} to Fe^{III} within said ferrous-ferric oxyhydroxy salt coupled to a stage of catalytic oxidation of the organic matter by reduction of the Fe^{III} to Fe^{II},
the redox potential of the pair S_{reduced}/S being greater than that of the pair Fe^{II}/Fe^{III} at the crystallographic sites of the Fe^{II}.

56. The process permitting the reduction of a substance S to a substance S_{reduced} according to claim 54, in which said LDH is used in association with a metal selected from Cu(II), Ag(I), Cd(II), Ni(II), Hg(II), Pb(II) and Mn(II), preferably Cu(II), in a proportion from 2 to 20% (w/w) relative to the total Fe.

57. The process permitting the reduction of a substance S to a substance S_{reduced} according to claim 54, in which said LDH is used in association with phosphate ions in a proportion of at least 1%.

58. The process according to claim 54, in which x is equal to 1 at the initial moment before the start-up of the oxidation-reduction process.

59. The process according to claim 50, said process taking place under conditions of anoxia.

60. The process according to claim 50, in which the anion is selected from carbonate, chloride, sulphate, fluoride, iodide, oxalate, methanoate.

61. The process according to claim 50, in which the substance S is selected from inorganic pollutants including nitrate, selenate, chromate, arsenate or from organic pollutants.

62. The process according to claim 50, in which the bacteria are selected from Shewanella putrefaciens, Geobacter sp.

63. The Process according to claim 50, in which the ferrous-ferric oxyhydroxy salt is prepared by bacterial synthesis, comprising:
culture of iron-reducing bacteria under conditions of anoxia in a suitable medium comprising:
Fe^{III}, in the form of an oxyhydroxide or a ferric oxyhydroxy salt of formula [Fe^{III}₃ₙO₆ₙH₄ₙ]ⁿ⁺[Aⁿ⁻, m H₂O]ⁿ⁻,
organic matter, including methanoate HCO₂⁻,
an anion Aⁿ⁻ if the anion is not HCO₃⁻,
in order to obtain a ferrous-ferric oxyhydroxy salt in which x varies in the range from 0.33 to 0.66.

64. Product constituted by at least one LDH, said LDH containing a divalent cation M²⁺ partially or completely substituted with Fe^{II}, and a trivalent cation T³⁺ optionally substituted with Fe^{III}, of the following general formula:
[M²⁺_{(z)}Fe^{II}_{(1-y-z)}T³⁺ₜFe^{III}_{(y-t)}O₂H₂]ⁿ⁺[(y/n)Aⁿ⁻,mH₂O]ⁿ⁻
in which:
¼<y<⅓, z<1−y and t<y, Aⁿ⁻ is an anion with charge n, n having the values 1, 2 or 3, m is an integer varying from 1 to 10,
and the ratio x=(y−t)/(1−z−t) can vary from 0 to 1, in crystalline form, the ratio of surface volume to specific volume being greater than 100, without a substantial change in the crystalline structure of said LDH.

65. The product according to claim 64, wherein m is an integer varying from 1 to 4.

66. The product according to claim 65, wherein m is an integer equal to 4.

67. The product according to claim 64, in which said LDH is constituted by a ferrous-ferric oxyhydroxy salt having the formula:
[Fe^{II}₃ₙ₍₁₋ₓ₎Fe^{III}₃ₙₓO₆ₙHₙ₍₇₋₃ₓ₎]ⁿ⁺[Aⁿ⁻,mH₂O]ⁿ⁻
in which Aⁿ⁻ is an anion with charge n, n having the values 1, 2 or 3, m is an integer varying from 1 to 10, and x is in the range from 0 to 1, in crystalline form, the ratio of surface volume to specific volume being greater than 100, without a substantial change in the crystalline structure of said ferrous-ferric oxyhydroxy salt.

68. The product according to claim 64, in which said LDH is used in association with a metal selected from Cu(II), Ag(I), Cd(II), Ni(II), Hg(II), Pb(II) and Mn(II), preferably Cu(II), in a proportion from 2 to 20% (w/w) relative to the total Fe.

69. The product according to claim 64, in which said LDH is used in association with phosphate ions in a proportion of at least 1%.

70. Product constituted by at least one support coated with at least one LDH, said LDH containing a divalent cation M²⁺ partially or completely substituted with Fe^{II}, and a trivalent cation T³⁺ optionally substituted with Fe^{III}, of the following general formula:
[M²⁺_{(z)}Fe^{II}_{(1-y-z)}T³⁺ₜFe^{III}_{(y-t)}O₂H₂]ⁿ⁺[(y/n)Aⁿ⁻,mH₂O]ⁿ⁻
in which:
¼<y<⅓, z<1−y and t<y, Aⁿ⁻ is an anion with charge n, n having the values 1, 2 or 3, m is an integer varying from 1 to 10,
and the ratio x=(y−t)/(1−z−t) can vary from 0 to 1, in crystalline form, the support being selected from sand, clay, polymer beads.

71. The product according to claim 70, wherein m is an integer varying from 1 to 4.

72. The product according to claim 71, wherein m is an integer equal to 4.

73. The product according to claim 70, in which said LDH is constituted by a ferrous-ferric oxyhydroxy salt having the formula:
[Fe^{II}₃ₙ₍₁₋ₓ₎Fe^{III}₃ₙₓO₆ₙHₙ₍₇₋₃ₓ₎]ⁿ⁺[Aⁿ⁻,mH₂O]ⁿ⁻,
in which Aⁿ⁻ is an anion with charge n, n having the values 1, 2 or 3, including the carbonate CO₃²⁻, m is an integer varying from 1 to 10, and x is in the range from 0 to 1, in crystalline form, the support being selected from sand, clay, polymer beads.

74. Product according to claim 70, in which said LDH is used in association with a metal selected from Cu(II), Ag(I), Cd(II), Ni(II), Hg(II), Pb(II) and Mn(II), preferably Cu(II), in a proportion from 2 to 20% (w/w) relative to the total Fe.

75. The product according to claim 70, in which said LDH is used in association with phosphate ions in a proportion of at least 1%.

76. The Product according to claim 70, characterized in that the ratio of the volume of the surface deposit of LDH to the volume of the support is between 1/100 and 1/10000.

77. The product according to claim 70, in which the LDH is the ferric oxyhydroxy salt of formula
[Fe^{III}₃ₙO₆ₙH₄ₙ]ⁿ⁺[Aⁿ⁻,mH₂O]ⁿ⁻
in which Aⁿ⁻ is an anion with charge n, n having the values 1, 2 or Sand m is an integer varying from 1 to 10, as obtained by implementation of the process comprising the stages of:
coprecipitation in solution of Fe^{II} and Fe^{III} ions in the presence of anions Aⁿ⁻, in the absence of oxygen, to obtain a ferrous-ferric hydroxy salt of formula
[Fe^{II}₂ₙFe^{III}ₙ(OH)₆ₙ]ⁿ⁺[Aⁿ⁻, mH₂O]ⁿ⁻,
complete and rapid oxidation by H₂O₂ or O₂, in solution or in air of said dry ferrous-ferric hydroxy salt after drying, to obtain a ferric oxyhydroxy salt of formula
[Fe^{III}₃ₙO₆ₙH₄ₙ]ⁿ⁺[Aⁿ⁻,mH₂O]ⁿ⁻,
drying of said ferric oxyhydroxy salt, in order to obtain a dry ferric oxyhydroxy salt, and
mixing of the dry ferric oxyhydroxy salt with the support, in order to obtain a support coated with the ferric oxyhydroxy salt.

78. Kit comprising:
at least one LDH, said LDH containing a divalent cation M²⁺ partially or completely substituted with Fe^{II}, and a trivalent cation T³⁺ optionally substituted with Fe^{III}, of the following general formula:
[M²⁺_{(z)}Fe^{II}_{(1-y-z)}T³⁺ₜFe^{III}_{(y-t)}O₂H₂]ⁿ⁺[(y/n)Aⁿ⁻,mH₂O]ⁿ⁻
in which:
¼<y<⅓, z<1−y and t<y, Aⁿ⁻ is an anion with charge n, n having the values 1, 2 or 3, m is an integer varying from 1 to 10,
and the ratio x=(y−t)/(1−z−t) can vary from 0 to 1, in crystalline form,
at least one support, selected from sand, clay, polymer beads,
to be used simultaneously, separately or spread over time, intended for the implementation of a process for pollution control of a medium to be treated.

79. The kit according to claim 78, wherein m is an integer varying from 1 to 4.

80. The kit according to claim 79, wherein m is an integer equal to 4.

81. The kit according to claim 78, in which the LDH is a ferrous-ferric oxyhydroxy salt and comprising:
at least one ferrous-ferric oxyhydroxy salt having the formula:
[Fe^{II}₃ₙ₍₁₋ₓ₎Fe^{III}₃ₙₓO₆ₙHₙ₍₇₋₃ₓ₎]ⁿ⁺[Aⁿ⁻,mH₂O]ⁿ⁻
in which Aⁿ⁻ is an anion with charge n, n having the values 1, 2 or 3, m is an integer varying from 1 to 10, and x is in the range from 0 to 1, in crystalline form,
at least one support, selected from sand, clay, polymer beads,
to be used simultaneously, separately or spread over time, intended for the implementation of a process for pollution control of a medium to be treated.

82. The kit according to claim 81, wherein m is an integer varying from 1 to 4.

83. The kit according to claim 82, wherein m is an integer equal to 4.

84. Method for the catalytic reduction of a substance S to a substance S_{reduced}, by means of a product according to claim 64, the redox potential of the pair S_{reduced}/S being greater than that of the pair Fe^{II}/Fe^{III} at the crystallographic sites of the Fe^{II}.

85. Method for the pollution control of a medium to be treated by means of a product according to claim 64.

86. Method for limiting the excessive proliferation of algae, including ulvae, by means of a product according to claim 64.
